(19) United States
(12) Patent Application Publication
(10) Pub. No.: US 2012/0197053 A1
(43) Pub. Date: Aug. 2, 2012

(54) SYSTEM AND METHOD FOR THE PRODUCTION OF LIQUID FUELS

(75) Inventors: Joel CANTRELL, College Station, TX (US); Duane Morrow, College Station, TX (US); Edward R. Peterson, Pearland, TX (US)

(73) Assignee: SYNFUELS INTERNATIONAL., INC., Dallas, TX (US)

(21) Appl. No.: 13/192,396

(22) Filed: Jul. 27, 2011

Related U.S. Application Data
(60) Provisional application No. 61/385,096, filed on Sep. 21, 2010.

(51) Int. Cl. C07C 2/08; B01J 19/00; C07C 7/00; C07C 5/03; C07C 5/09

(52) U.S. Cl. 585/251; 585/517; 585/255; 585/329; 422/187

(57) ABSTRACT

A method of producing liquid fuels by providing an olefin feed containing at least one C2-C20 olefin, oligomerizing a part of the feed in the presence of a first catalyst to form a first product comprising oligomers of the at least one olefin, and oligomerizing a portion of the first product in the presence of a second catalyst to produce a second product. A system of producing liquid hydrocarbons, the system including a first reactor configured to provide a first product by oligomerizing, in the presence of a first catalyst, at least a portion of an olefin feed comprising at least one olefin, a separator configured to provide an unreacted olefin-reduced first product by separating unreacted olefin from the first product, and a second reactor configured to provide a second product by oligomerizing, in the presence of a second catalyst, at least a portion of the unreacted olefin-reduced first product.

### CROSS-REFERENCE TO RELATED APPLICATIONS

This application claims the benefit under 35 U.S.C. §119(e) of U.S. Provisional Patent Application Ser. No. 61/385,096 filed Sep. 21, 2010, which is hereby incorporated herein by reference in its entirety.

### STATEMENT REGARDING FEDERALLY SPONSORED RESEARCH OR DEVELOPMENT

Not applicable.

### BACKGROUND

1. Field of the Invention

This invention relates to the production of liquid fuels. More specifically, this invention relates to converting non-hydrocarbon liquids into hydrocarbon liquids. Still more specifically, this invention relates to converting non-hydrocarbon components into designable liquid fuels selected from the group consisting of kerosene, jet fuel, gasoline, diesel and combinations thereof, wherein the designable liquid fuels comprise desired amounts of specific components, such as, but not limited to, aromatics.

2. Background of the Invention

The ever-increasing demand for liquid fuels from petroleum is predicted to exceed the supply in the future. Additionally, the production of liquid fuels from petroleum raises environmental concerns, for which ever more stringent regulations are enacted. Thus, alternative methods of obtaining liquid fuels are sought.

Ethylene and other olefins can be formed via conversion of various readily-available compounds, including, for example, oxygenates. For example, olefins can be produced via, among others, processes such as the dehydration of alcohols including methanol, ethanol, propanol, butanol and heavier mono-alcohols, polyols, oxygenates, aldehydes, ketones and ethers; the hydrogenation of acetylenic compounds; the hydration of calcium carbide; and the dehydrohalogenation of chlorinated hydrocarbons.

The desired composition of a liquid fuel depends on the ultimate application envisioned. For example, it is mandated that jet fuels comprise a specific volume percent of aromatic compounds. The required specific volume percent of aromatic compounds may change as various regulations are instituted. Thus, systems and methods of producing liquid fuels from non-petroleum sources that enable the creation of a liquid fuel having a desired composition, which desired composition may change in response to new regulations and etc., thus allowing for alterations in the product fuel produced therefrom and thereby are very desirable.

Accordingly, there is a need in the industry for a system and method to produce fuels, particularly liquid fuels, from non-petroleum materials. Desirably, such a system and method allows for the production of liquid fuels from non-hydrocarbon materials. Also desirable, are a system and method for producing liquid fuels that can be readily tailored for the creation of a liquid fuel having a desired composition, e.g. a desired aromaticity, average molecular weight, boiling point range, and/or etc. Further desirable is the production of liquid fuels from a sustainable source, such as alcohols produced by fermentation of biomass.

### SUMMARY

Herein disclosed are a system and method for converting non-hydrocarbon materials to liquid fuels. In embodiments, the method comprises providing an olefin feed comprising at least one olefin selected from the group consisting of C2-C20 olefins; oligomerizing at least a part of the olefin feed in the presence of a first oligomerization catalyst to form a first oligomerization product comprising oligomers of the at least one olefin; and subjecting at least a portion of the first oligomerization product to oligomerization in the presence of a second oligomerization catalyst to produce a second oligomerization product. The method may further comprise hydrogenating at least a portion of the second oligomerization product to produce a hydrogenation product, wherein the average degree of saturation of the hydrogenation product is greater than the average degree of saturation of the second oligomerization product. In embodiments, the hydrogenation product comprises primarily hydrocarbons boiling in a boiling point range selected from the group consisting of the gasoline boiling point range, the naphtha boiling point range, the kerosene boiling point range and the diesel boiling point range. The hydrogenation product may comprise from about 20 to about 50 volume percent aromatic compounds, from about 8 to about 12 volume percent aromatic compounds, from about 4 to about 8 volume percent aromatic compounds, or from about 12 to about 20 volume percent aromatic compounds.

In embodiments, providing the olefin feed comprises performing one or more process selected from the group consisting of alcohol dehydration processes and acetylenic compound hydrogenation processes. In embodiments, providing the olefin feed comprises catalytically converting an oxygen-containing compound in an oxygenate feed to an olefin. The olefin feed may comprise ethylene and providing the olefin feed may further comprise separating a stream enriched in ethylene from the product of the catalytic conversion of an oxygenate feed. The method may further comprise separating a stream comprising unreacted oxygen-containing compounds from an ethylene-reduced remainder of the product of the catalytic conversion of the oxygenate feed. The method may further comprise producing additional olefin from the stream comprising unreacted oxygen-containing compounds; and/or separating a liquid hydrocarbon phase, a water phase, or both from the ethylene-reduced remainder of the product of the catalytic conversion of the oxygenate feed. At least a portion of the liquid hydrocarbon phase may be subjected to the second oligomerization.

In embodiments, the method further comprises separating an unreacted olefin stream comprising unreacted olefin from the first oligomerization product prior to the second oligomerization. The method may further comprise recycling at least a portion of the unreacted olefin stream to the first oligomerization.

In embodiments, the method further comprises separating the second oligomerization product into one or more streams selected from the group consisting of a stream enriched in olefins boiling in the gasoline boiling point range, a stream enriched in naphtha, and a stream enriched in C2 through C20 olefins relative to the second oligomerization product. At least a portion of the stream enriched in olefins boiling in the gasoline boiling point range may be subjected to the first oligomerization, the second oligomerization, or both. In embodiments, at least a portion of the stream enriched in C2 through C20 olefins is subjected to hydrogenation to produce a hydrogenation product, wherein the average degree of saturation of the hydrogenation product is greater than the average degree of saturation of the stream enriched in C2 through C20 olefins; and/or subjecting at least a portion of the stream enriched in C2 through C20 olefins to the second oligomerization. At least a portion of the hydrogenation product may be recycled to the second oligomerization.

In embodiments, the method further comprises subjecting a portion of the first oligomerization product to oligomerization in the presence of a third oligomerization catalyst to produce a third oligomerization product. In embodiments, the method further comprises separating an unreacted olefin stream comprising unreacted olefin from the third oligomerization product; or subjecting at least a portion of the third oligomerization product at least a portion of the second oligomerization product, or both to hydrogenation to produce one or more hydrogenation products, wherein the average degree of saturation of the one or more hydrogenation products is greater than the average degree of saturation of the oligomerization product prior to hydrogenation. At least a portion of the unreacted olefin stream may be recycled to the third oligomerization. At least a portion of the one or more hydrogenation products may be subjected to the second oligomerization.

In embodiments, the first oligomerization catalyst comprises solid acid catalyst such as a silica aluminate, more specifically ZSM-5. In embodiments, the first oligomerization catalyst comprises nickel activated ZSM-5. In embodiments, the second oligomerization catalyst comprises a solid acid catalyst such as silica aluminate, more specifically a zeolite such as ZSM-5.

In embodiments, the first oligomerization product comprises primarily butene and the second oligomerization product comprises oligomers of butene.

Also disclosed herein is a system comprising a first oligomerization reactor configured to oligomerize at least a portion of an olefin feed comprising at least one olefin in the presence of a first oligomerization catalyst, providing a first oligomerization product; a separator configured to separate unreacted olefin from the first oligomerization product, providing an unreacted olefin reduced first oligomerization product; and a second oligomerization reactor configured to oligomerize at least a portion of the unreacted olefin reduced first oligomerization product in the presence of a second oligomerization catalyst, providing a second oligomerization product. The olefin feed may comprise primarily ethylene or primarily butene.

In embodiments, the first oligomerization catalyst is operable to convert the at least one olefin primarily to dimers thereof. The first oligomerization catalyst may comprise nickel activated ZSM-5.

In embodiments, the second oligomerization catalyst is operable to convert at least a portion of oligomers in the first oligomerization product to longer chain olefins. The second oligomerization catalyst may comprise naked ZSM-5.

In embodiments, the system further comprises a hydrogenation reactor configured to catalytically reduce the degree of unsaturation of the second oligomerization product, thus providing a hydrogenated product, and may further comprise recycling at least a portion of the hydrogenated product, at least a portion of the second oligomerization product, or both, to the first oligomerization reactor, the second oligomerization reactor, or both, whereby a hydrocarbon product having a desired composition may be obtained. The desired composition may include an aromatics content in the range of from about 4 to about 50 volume percent, from about 4 to about 8 volume percent, from about 8 to about 12 volume percent, or from about 12 to about 20 volume percent. The desired composition may comprise primarily diesel, kerosene, gasoline, or naphtha boiling point range hydrocarbons.

In embodiments, the system further comprises a second separator configured to separate one or more streams selected from the group consisting of a stream comprising primarily naphtha, a stream comprising primarily gasoline, and a stream comprising primarily C2-C30 olefins from the second oligomerization product. One or more recycle lines may be configured for recycle of at least a portion of a stream comprising primarily gasoline to the first oligomerization reactor, to the second oligomerization reactor, or to both. The system may further comprise a recycle line configured for recycle of at least a portion of a stream comprising primarily C2-C30 olefins to the second oligomerization reactor.

In embodiments, the system further comprises a third oligomerization reactor configured to oligomerize at least a portion of an olefin feed comprising at least one olefin in the presence of a third oligomerization catalyst, providing a third oligomerization product; and/or a second separator configured to remove unreacted olefins from the third oligomerization product, providing an unreacted olefin reduced third oligomerization product. The third oligomerization catalyst may comprise ZSM-5. In embodiments, the system further comprises a recycle line configured for recycle of at least a portion of the unreacted olefins removed via the second separator to the second oligomerization reactor. The system may further comprise one or more hydrogenation reactors configured to catalytically reduce the degree of unsaturation of the second oligomerization product, the third oligomerization product or both, providing one or more hydrogenated products. The system may further comprise one or more recycle lines configured for recycle of at least a portion of the one or more hydrogenated products to the third oligomerization reactor.

The system may further comprise a dehydration reactor configured to produce the olefin feed. In embodiments, the dehydration reactor is operable at a pressure greater than an operating pressure of the first oligomerization reactor, and the system comprises no compressor between the dehydration reactor and the first oligomerization reactor.

Thus, embodiments described herein comprise a combination of features and advantages intended to address various shortcomings associated with certain prior devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the invention, reference will now be made to the accompanying drawings in which:

FIG. 1 illustrates a process for making liquid fuel according to an embodiment of this disclosure;

FIG. 2 illustrates a process for making liquid fuel according to another embodiment of this disclosure; and

FIG. 3 illustrates a process for making liquid fuel according to another embodiment of this disclosure.

### DETAILED DESCRIPTION

Herein disclosed are a system and process for producing liquid fuels from non-hydrocarbon materials. Via the disclosed system and method, a desired liquid fuel can be produced, i.e. a liquid fuel comprising primarily hydrocarbons in the gasoline, diesel, jet fuel and/or kerosene boiling point range and/or containing a desired amount of various components, such as, but not limited to, aromatics.

System for the Production of Liquid Fuels. Herein disclosed is a system for producing liquid fuels from non-petroleum materials. In embodiments, the disclosed system allows for the production of liquid fuels from non-hydrocarbon materials. In embodiments, a system of this disclosure comprises at least one reactor configured for the production of olefins (e.g. from oxygen-containing compounds) and one or more oligomerization reactors configured for the production of oligomers and other hydrocarbons from the olefins. In embodiments, a system of this disclosure comprises a plurality of oligomerization reactors. The system may further comprise one or more reactors (e.g. hydrogenation reactors) configured for the production of various rearranged (e.g. hydrogenated) hydrocarbons from the oligomers and other hydrocarbons produced in the at least one oligomerization reactor or the plurality of oligomerization reactors. The system may further comprise one or more separators (e.g. phase separators), one or more gas cleanup apparatus, one or more pumps, and/or one or more compressors as described in more detail hereinbelow.

System Comprising Single Oligomerization Reactor. In embodiments, a system of this disclosure comprises at least one olefin production reactor and at least one oligomerization reactor. The system may further comprise at least one reactor configured for the production of various rearranged (e.g. hydrogenated) hydrocarbons from the oligomers and other hydrocarbons.

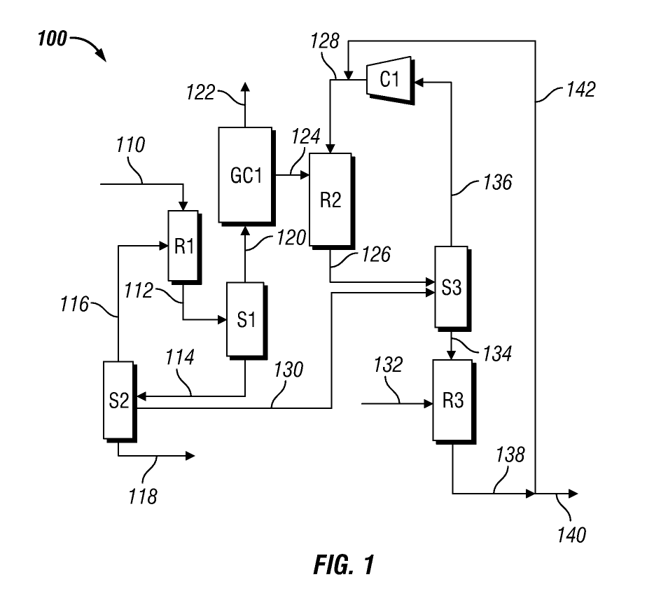

A system for the production of liquid hydrocarbons from non-hydrocarbon components according to this disclosure will now be described with reference to FIG. 1, which depicts a system 100 comprising a single olefin production reactor, first reactor R1, a single oligomerization reactor, second reactor R2, and a single hydrogenation reactor, third reactor R3. The disclosed system may further comprise one or more separators, gas cleanup apparatus, compressors, and/or pumps, as further discussed hereinbelow. System 100 further comprises separators S1, S2, and S3, compressor C1, and gas cleanup apparatus GC1. Each of these components will be described in more detail hereinbelow.

System 100 comprises first reactor R1, which is an olefin production reactor configured for converting oxygen-containing compounds introduced thereto via line 110 primarily to olefins. First reactor R1 may be configured to convert, primarily to olefins, a feed comprising one or more oxygen-containing components selected from C1 and higher alcohols, water, C2 and similar oxygenates including, but not limited to, acids, ethers, epoxides, aldehyes and other oxygen containing compounds. First reactor R1 contains a catalyst that is capable of catalyzing the conversion of oxygen containing compounds (i.e. ‘oxygenates’) primarily to olefins. Any suitable catalyst known in the art may be utilized. In embodiments, first reactor R1 comprises an aluminum oxide catalyst. First reactor R1 may be configured to convert, primarily to olefins, a feed comprising components that are liquid either in pure form or in mixture at conditions of higher pressure and/or lower temperature than the operating conditions of first reactor R1. First reactor R1 may be operable to provide a product, removable therefrom via line 112, comprising at least 50% of the available carbon as olefin product and more preferably at least 90% of the available feed carbon as olefin product and most preferably at least 95% of the available feed carbon as olefin product.

First reactor R1 may be configured for the production of primarily C2 and/or C3 olefins via one or more process selected from: dehydration of alcohols including, without limitation, methanol, ethanol, propanol, butanol and/or heavier mono-alcohols, polyols, oxygenates, aldehydes, ketones and ethers; hydrogenation of acetylenic compounds formed by known methods including, without limitation, pyrolysis of natural gas and other hydrocarbons, hydration of calcium carbide, dehydrohalogenation of monochlorinated and polychlorinated hydrocarbons, and other known methods of producing compounds containing olefin functionality.

The operating conditions of first reactor R1 will depend on the selection of catalyst and the composition of the feed stream introduced thereto via line 110. Thus, first reactor R1 may be operated as known in the art to provide a stream comprising primarily olefins. In embodiments, first reactor R1 is operated at an operating pressure in the range of from about 15 psia to about 800 psia (103 kPa to 5500 kPa), from about 50 psia to about 400 psia (340 kPa to 2800 kPa), or from about 250 psia to about 300 psia (1700 kPa to 2100 kPa). In embodiments, first reactor R1 is operated at an operating temperature in the range of from about 50° C. to about 400° C., from about 100° C. to about 375° C., or from about 250° C. to about 350° C.

System 100 further comprises first separator S1. First separator S1 may be a phase separator configured to separate a liquid phase from a gas phase. First separator S1 may be any suitable phase separator known in the art to be suitable for separating the primarily olefin product of first reactor R1 into desired liquid and gas phases. First separator S1 is fluidly connected via line 112 to first reactor R1, whereby a stream comprising primarily olefins formed within first reactor R1 may be introduced into first separator S1. First reactor R1 may be configured to provide a product, introducible into first separator S1 via line 112, that comprises primarily olefins, but may further comprise one or more components selected from alcohols, ethers, aldehydes, such as acetaldehyde, water, carbon monoxide, carbon dioxide, hydrogen, and C1 through C16 hydrocarbons. In embodiments, phase separator S1 is configured to separate a liquid phase comprising primarily water, heavier oxygenates and C4+ hydrocarbons, removable therefrom via line 114, from a gas phase, which is removable therefrom via a line 120.

First separator S1 may be configured to provide a vapor phase, removable therefrom via line 120, that comprises primarily olefins. In embodiments, first separator S1 is configured to provide a vapor phase comprising at least 20% of the available olefins, at least 75% of the available olefins, or at least or more than 90% of the available olefins. In embodiments, the feed comprises ethanol and first separator S1 is configured to provide a vapor phase comprising at least 20 volume percent of the ethylene introduced thereto, at least 75 volume percent of the ethylene introduced thereto, or at least 90 volume percent of the ethylene introduced thereto.

System 100 further comprises second separator S2, configured to separate the produced liquid from first separator S1 into a water phase, a liquid hydrocarbon phase, and a light vapor stream. Second separator S2 is fluidly connected via line 114 with first separator S1. Second separator S2 may be any separator known in the art to be suitable for separating a liquid hydrocarbon stream from a light vapor stream and a water phase. In embodiments, second separator S2 is selected from partial flash drums, single stage flash drums, multiple stage distillation towers and similar devices operable to separate the liquid removed from first separator S1 into desired water, liquid hydrocarbon and light vapor streams. A line 118 is configured for the removal of a water phase from second separator S2. A line 130 is configured for the removal of a liquid hydrocarbon phase from second separator S2. In embodiments, second separator S2 is operable to provide a liquid hydrocarbon phase, removable therefrom via line 130, that comprises at least 50% volume percent hydrocarbons, at least 80 volume percent hydrocarbons, or at least 95 volume percent hydrocarbons. In embodiments, line 130 fluidly connects second separator S2 with a third separator S3 (discussed further hereinbelow), whereby hydrocarbons in the liquid hydrocarbon phase separated from separator S2 may be introduced into second reactor R2 and/or third reactor R3 (discussed further hereinbelow) for further processing.

A line 116 is configured for removal of a light vapor stream from second separator S2. Second separator S2 may be configured to provide a light vapor stream comprising oxygenates. In embodiments, second separator S2 is configured to provide a vapor phase, removable therefrom via line 116, that comprises at least 5 volume percent oxygenates, at least 10 volume percent oxygenates, or at least 50 volume percent oxygenates. In such embodiments, line 116 may fluidly connect second separator S2 with first reactor R1, whereby oxygenates in the light vapor stream may be returned to first reactor R1 for reprocessing. System 100 further comprises gas cleanup apparatus GC1, configured for removal of non-olefin gas species from the vapor phase removed from phase separator S1 via line 120. A line 122 is configured for removal of primarily non-olefin components from GC1 and a line 124 is configured for removal of purified olefin product from GC1. Gas cleanup apparatus GC1 may be configured for substantially complete removal of non-olefins from a vapor introduced thereto, providing a non-olefin product extractable therefrom via line 122 and a purified olefin product extractable therefrom via line 124. In embodiments, GC1 is configured to provide a product, removable therefrom via line 124, comprising less than about 5 volume percent non-olefin components, less than about 0.5 volume percent non-olefin components, or less than about 0.01 volume percent non-olefin components.

System 100 further comprises second reactor R2, which is an oligomerization reactor configured to convert olefins to larger olefins, aromatics and/or other hydrocarbons by the processes of oligomerization and/or aromatization. Second reactor R2 is fluidly connected with gas cleanup apparatus GC1 via line 124, whereby a purified olefin stream extracted from gas cleanup apparatus GC1 may be introduced into second reactor R2. Second reactor R2 may be any reactor known in the art to be suitable for, and may contain any catalyst known in the art to be operable to catalyze, the conversion of olefins to larger olefins, aromatics and/or other hydrocarbons. For example, in embodiments, second reactor R2 contains nickel activated solid acid catalyst such as a silica aluminate, for example ZSM-5. In embodiments, second reactor R2 contains a zeolite catalyst, such as, but not limited to, ZSM-5 (e.g. naked ZSM-5). Second reactor R2 may be operable to provide a product comprising components selected from oligomers of the olefins introduced thereto, mono- and multiply substituted aromatic compounds, isomerized olefins, saturated hydrocarbons, naphthenes, saturated and unsaturated cyclic hydrocarbons, and/or unreacted feed components. In embodiments, second reactor R2 is configured to provide a product, extractable therefrom via line 126, comprising about 70 volume percent product characterizable as naphtha and/or gasoline, about 20 volume percent product characterizable as middle distillate, and/or about 10 volume percent product characterizable as heavier oil. In embodiments, second reactor R2 is configured to provide a product, extractable therefrom via line 126, comprising about 15 volume percent product characterizable as naphtha and/or gasoline, about 70 volume percent product characterizable as middle distillate, and/or about 15 volume percent product characterizable as heavier oil. In other embodiments, second reactor R2 is configured to provide a product, extractable therefrom via line 126, comprising 10 volume percent product characterizable as naphtha and/or gasoline, 15 volume percent product characterizable as middle distillate, and/or 75 volume percent product characterizable as heavier oil. In embodiments, second reactor R2 is configured to provide a product that comprises primarily butene or primarily butene and hexene. In embodiments, second reactor R2 is configured to provide a product that comprises primarily components selected from the group consisting of butenes, hexenes, octenes, decenes, and heavier mono-olefins, diolefins, naphthenes, and aromatic compounds.

The operating conditions of second reactor R2 will depend on the selection of catalyst and the composition of the feed stream introduced thereto via line 124. Thus, second reactor R2 may be operated as known in the art to provide a stream comprising primarily oligomers of the olefins in the feed thereto. In embodiments, second reactor R2 is operated at a temperature in the range of from about 150° C. to about 400° C., from about 150° C. to about 350° C., or from about 175° C. to about 300° C. In embodiments, second reactor R2 is operated at a pressure in the range of from about 25 psi (172.4 kPa) to about 1000 psi (6894.8 kPa), from about 150 psi (1034 kPa) to about 600 psi (4140 kPa), or from about 200 psi (1380 kPa) to about 400 psi (2760 kPa).

In embodiments, the operating pressure of first reactor R1 is greater than the operating pressure of second reactor R2. In such embodiments, the system may not comprise a compressor for conveying the gaseous products from reactor R1 to reactor R2. That is, in embodiments, dehydration is performed in first reactor R1 at a pressure above the oligomerization pressure of second reactor R2.

System 100 further comprises third separator S3, configured for separating a liquid hydrocarbon stream from a vapor phase comprising unreacted and/or lower molecular weight olefinic compounds. Third separator S3 is fluidly connected with second reactor R2 via line 126, whereby product from second reactor R2 may be introduced into third separator S3. Third separator S3 may be any suitable phase separator known in the art to separate a liquid hydrocarbon stream from unreacted and/or lower molecular weight, gaseous olefins. A line 134 is configured for the removal of liquid hydrocarbons from third separator S3, while a line 136 is configured for removal therefrom of a gaseous/vapor phase comprising unreacted and/or lower molecular weight olefins. Third separator S3 may be fluidly connected via line 136, a first compressor C1, and line 128 with an inlet of second reactor R2, whereby unreacted and/or lower molecular weight olefins removed from third separator S3 via line 136 may be compressed and reintroduced into second reactor R2 for further processing. Compressor C1 may be configured to pressurize the gas to a pressure of from about 200 to about 7000 kPa. In embodiments, the liquid product removed from third separator S3 via line 134 comprises at least 90 volume percent hydrocarbons, at least 95 volume percent hydrocarbons or at least 99 volume percent hydrocarbons. Depending on the desired product (primarily gasoline, jet fuel, and/or diesel), in embodiments, the liquid product removed from third separator S3 via line 134 comprises primarily hydrocarbons from C7 to C16, from C10 to C16, or from C7 to C10, depending on whether jet fuel, diesel, or gasoline is being produced.

System 100 further comprises third reactor R3, which is a hydrogenation reactor configured for and/or containing catalyst operable to catalyze the saturation of hydrocarbons introduced thereto. Third reactor R3 is fluidly connected with third separator S3 via line 134. Third reactor R3 may be any reactor known in the art to be suitable for the conversion of unsaturated compounds (e.g. olefins and other unsaturated compounds) to more saturated hydrocarbons. For example, by way of non-limiting example, third reactor R3 may contain Pd impregnated ZSM-5 catalyst or Ni/Mo on alumina catalyst. A line 132 is configured to introduce hydrogen into third reactor R3. Third reactor R3 is operable at conditions of WHSV, temperature and pressure effective for the conversion of at least a portion of the unsaturated components (e.g. a portion of the olefinic content) of the feed introduced thereto via line 134 into more saturated or substantially entirely saturated product.

In embodiments, third reactor R3 is operated at a temperature in the range of from about 150° C. to about 350° C., from about 200° C. to about 300° C., or from about 250° C. to about 270° C. In embodiments, third reactor R3 is operated at a pressure in the range of from about 2100 kPa to about 6900 kPa, from about 2700 kPa to about 5500 kPa, or from about 3400 kPa to about 4200 kPa. Depending on the catalyst chosen, the WHSV can range from about 0.1 to 10, or from about 0.5 to about 1.0 hr⁻¹.

Third reactor R3 is configured to provide a product, extractable therefrom via line 138, comprising saturated hydrocarbons, aromatic hydrocarbons and/or olefinic hydrocarbons, wherein the degree of unsaturation (or the olefinic content) of the product extracted via line 138 is less than or equal to the degree of unsaturation (or the olefinic content) of the feed introduced thereto via line 134. In embodiments, system 100 further comprises a line 142 configured for recycle of at least a portion of the product removed from third reactor R3 via line 138 to second reactor R2. The product recycled in this manner may serve as a substantially non-reactive component, thus moderating various second reactor R2 and third reactor R3 operating conditions, such as, but not limited to, temperature, pressure and WHSV. In such embodiments, a line 140 may be configured for the removal of the non-recycled product from system 100.

System 100 is configured to provide a product, removable therefrom via line 140, comprising liquid hydrocarbons. In embodiments, system 100 is operable to provide a product comprising naphtha, gasoline, middle distillate and heavier oil.

Although three separators are depicted and described in the embodiment of FIG. 1, it is to be understood that more or fewer separators may be utilized in some embodiments. Also, other apparatus known in the art may be incorporated into the system, as will be apparent to those of skill in the art.

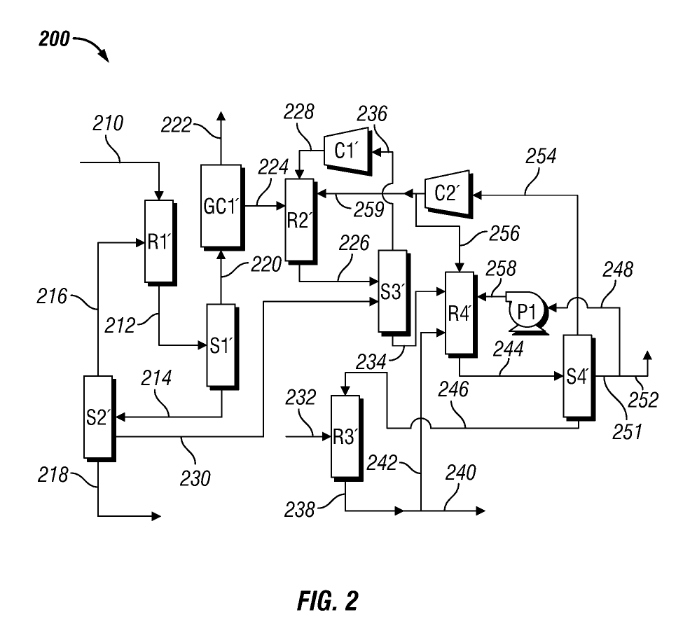

System Comprising Two Oligomerization Reactors. In embodiments, a system of this disclosure comprises a plurality of oligomerization reactors. For example, another system for the production of liquid fuel from non-hydrocarbon components according to this disclosure will now be described with reference to FIG. 2. System 200 comprises reactors R1′, R2′ and R3′ and R4′, wherein first reactor R1′ is an olefin production reactor, second reactor R2′ is a primary oligomerization reactor, third reactor R3′ is a hydrogenation reactor and fourth reactor R4′ is a secondary oligomerization reactor. System 200 further comprises separators SP, S2′, S3′ and S4′, compressors C1′ and CT, gas cleanup apparatus GC1′, and pump P1′. Each of these components will be described in more detail hereinbelow.

System 200 comprises first reactor R1′, which is an olefin production reactor configured for converting oxygen-containing compounds introduced thereto via line 210 primarily to olefins. First reactor R1′ may be configured to convert, primarily to olefins, a feed comprising one or more oxygen-containing components selected from C1 and higher alcohols, water, C2 and similar oxygenates including, but not limited to, acids, ethers, epoxides, aldehyes and other oxygen containing compounds. First reactor R1′ contains a catalyst that is capable of catalyzing the conversion of oxygen containing compounds (i.e. ‘oxygenates’) primarily to olefins. Any suitable catalyst known in the art may be utilized. In embodiments, first reactor R1′ comprises an aluminum oxide catalyst. First reactor R1′ may be configured to convert, primarily to olefins, a feed comprising components that are liquid either in pure form or in mixture at conditions of higher pressure and/or lower temperature than the operating conditions of first reactor R1′. First reactor R1′ may be operable to provide a product, that is removable therefrom via line 212, comprising at least 50% of the available carbon as olefin product, at least 90% of the available feed carbon as olefin product, or at least 95% of the available feed carbon as olefin product.

First reactor R1′ may be configured for the production of C2 and/or C3 olefins via one or more process selected from: dehydration of alcohols including, without limitation, methanol, ethanol, propanol, butanol and/or heavier mono-alcohols, polyols, oxygenates, aldehydes, ketones and ethers; hydrogenation of acetylenic compounds formed by known methods including, without limitation, pyrolysis of natural gas and other hydrocarbons, hydration of calcium carbide, dehydrohalogenation of monochlorinated and polychlorinated hydrocarbons, and other known methods of producing compounds containing olefin functionality from C2 through C3.

The operating conditions of first reactor R1′ will depend on the selection of catalyst and the composition of the feed stream introduced thereto via line 210. Thus, first reactor R1′ may be operated as known in the art to provide a stream comprising primarily olefins. In embodiments, first reactor R1′ is operated at an operating temperature in the range of from about 50° C. to 400° C., from about 100° C. to about 375° C., or from about 250° C. to about 350° C. In embodiments, first reactor R1′ is operated at an operating pressure in the range of from about 15 psia to about 800 psia (103 kPa to 5500 kPa), from about 50 psia to about 400 psia (340 kPa to 2800 kPa), or from about 250 psia to about 300 psia (1700 kPa to 2100 kPa).

System 200 further comprises first separator SP. First separator SP may be a phase separator configured to separate a liquid phase from a gas phase. First separator SP may be any suitable phase separator known in the art to be suitable for separating the primarily olefin product of first reactor R1′ into desired liquid and gas phases. First separator SP is fluidly connected via line 212 to first reactor R1′, whereby a stream comprising primarily olefins formed within first reactor R1′ may be introduced into first separator SP. First reactor R1′ may be configured to provide a product, introducible into first separator SP via line 212, that comprises primarily olefins, but may further comprise one or more components selected from alcohols, ethers, acetaldehyde, aldehydes, water, carbon monoxide, carbon dioxide, hydrogen, and C1 through C16 hydrocarbons. In embodiments, phase separator S1′ is configured to separate a liquid phase comprising primarily water, heavier oxygenates and C4+ hydrocarbons, which is removable therefrom via line 214, from a gas phase, which is removable via a line 220. First separator S1′ may be configured to provide a vapor phase comprising primarily olefins. In embodiments, first separator S1′ is configured to provide a vapor phase, removable therefrom via line 220, that comprises at least 20% of the available olefins, at least 75% of the available olefins, or at least 90% of the available olefins. In embodiments, first separator S1′ is configured to provide a vapor phase comprising at least 20 volume percent of the ethylene introduced thereto, at least 75 volume percent of the ethylene introduced thereto, or at least 90 volume percent of the ethylene introduced thereto.

System 200 further comprises second separator S2′, configured to separate the produced liquid from first separator S1′ into a water phase, a liquid hydrocarbon phase, and a light vapor stream. Second separator S2′ is fluidly connected via line 214 with first separator S1′. Second separator S2′ may be any separator known in the art to be suitable for separating a liquid hydrocarbon stream from a light vapor stream and a water phase. In embodiments, second separator S2′ is selected from partial flash drums, single stage flash drums, multiple stage distillation towers and similar devices operable to separate the liquid removed from first separator S1′ into desired water, liquid hydrocarbon and light vapor streams. A line 218 is configured for the removal of a water phase from second separator S2′. A line 230 is configured for the removal of a liquid hydrocarbon phase from second separator S2′. In embodiments, second separator S2′ is operable to provide a liquid hydrocarbon phase, removable therefrom via line 230, that comprises at least 50 volume percent hydrocarbons, at least 80 volume percent hydrocarbons, or at least 95 volume percent hydrocarbons. In embodiments, line 230 fluidly connects second separator S2′ with a third separator S3′ (discussed further hereinbelow), whereby hydrocarbons in the liquid hydrocarbon phase separated from separator S2′ may be introduced into reactor R2′ and/or reactor R4′ (discussed further hereinbelow) for further processing.

A line 216 is configured for the removal of a light vapor stream from second separator S2′. Second separator S2′ may be configured to provide a light vapor stream comprising oxygenates. In embodiments, second separator S2′ is configured to provide a vapor phase, removable therefrom via line 216, that comprises at least 5 volume percent oxygenates, at least 10 volume percent oxygenates, or at least 50 volume percent oxygenates. In such embodiments, line 216 may fluidly connect second separator S2′ with first reactor R1′, whereby oxygenates in the light vapor stream may be returned to first reactor R1′ for reprocessing.

System 200 further comprises gas cleanup apparatus GC1′, configured for removal of non-olefin gas species from the vapor phase removed from phase separator SP outlet line 220. Gas cleanup apparatus GC1′ may be configured for substantially complete removal of non-olefins from a vapor introduced thereto. In embodiments, GC1′ is configured to provide a product, removed therefrom via line 224, comprising less than about 5 volume percent non-olefin components, less than about 0.5 volume percent non-olefin components, or less than about 0.01 volume percent non-olefin components. A line 222 is configured for removal of primarily non-olefin components from GC1′.

System 200 further comprises second reactor R2′, which is a first oligomerization reactor configured for converting olefins primarily to oligomers, while minor amounts of aromatics and/or other hydrocarbons may also be produced. Second reactor R2′ is fluidly connected with gas cleanup apparatus GC1′ via line 224, whereby a purified olefin stream extracted from gas cleanup apparatus GC1′ may be introduced into second reactor R2′. Second reactor R2′ may be any reactor known in the art to be suitable for, and may contain any catalyst known in the art to be operable to catalyze, the conversion of olefins to dimers, trimers, or other oligomers. For example, in embodiments, second reactor R2′ contains a zeolite catalyst. In embodiments, second reactor R2′ comprises nickel activated or nickel imbibed ZSM-5 catalyst. In embodiments, second reactor R2′ comprises nickel imbibed ZSM-5 catalyst comprising from about 0.01 weight percent to about 15 weight percent nickel, from about 1 weight percent to about 10 weight percent nickel, or from about 2 weight percent to about 8 weight percent nickel. Second reactor R2′ may be operable to provide a product comprising components selected from dimers and oligomers of the olefins introduced thereto, mono- and multiply substituted aromatic compounds, isomerized olefins, saturated hydrocarbons, naphthenes, saturated and unsaturated cyclic hydrocarbons, and/or unreacted feed components. In embodiments, second reactor R2′ is configured to provide a product, extractable therefrom via line 226, comprising ethylene, propylene, butylenes, pentenes, hexenes and/or higher mono-olefins. Desirably, second reactor R2′ is configured to provide a product, extractable therefrom via line 226, comprising at least 10, 15, 20, 30, 40, 50, or 60 volume percent butylenes and/or heavier olefins.

The operating conditions of second reactor R2′ will depend on the selection of catalyst and the composition of the feed stream introduced thereto via line 224. Thus, second reactor R2′ may be operated as known in the art to provide a stream comprising primarily dimers of the olefins in the feed thereto (e.g. butylenes from an ethylene feed). In embodiments, second reactor R2′ is operated at a temperature in the range of from about 80° C. to about 400° C., from about 100° C. to about 280° C., or from about 150° C. to about 250° C.

In embodiments, second reactor R2′ is operated at a pressure in the range of from about 200 psia (1380 kPa) to about 600 psia (4137 kPa), from about 1650 kPa to about 2400 kPa, or from about 1790 kPa to about 2210 kPa. In embodiments, the operating pressure of first reactor R1′ is greater than the operating pressure of second reactor R2′. In such embodiments, the system may not comprise a compressor for conveying the gaseous products from reactor R1′ to reactor R2′. That is, in embodiments, dehydration is performed in first reactor R1′ at a pressure above the oligomerization pressure of second reactor R2′.

In embodiments, second reactor R2′ is operated at a weight hourly space velocity (WHSV) in the range of from about 0.001 to about 10, from about 0.1 to about 2.0, or from about 0.25 to about 1.0.

System 200 further comprises third separator S3′, configured for separating a liquid hydrocarbon stream from a vapor phase comprising primarily unreacted olefinic compounds. Third separator S3′ is fluidly connected with second reactor R2′ via line 226, whereby product from second reactor R2′ may be introduced into third separator S3′. Third separator S3′ may be any suitable phase separator known in the art to separate a liquid hydrocarbon stream from unreacted, gaseous olefins. A line 234 is configured for the removal of liquid hydrocarbons from third separator S3′, while a line 236 is configured for removal therefrom of a gaseous/vapor phase comprising unreacted olefins. Third separator S3′ may be configured to provide a liquid, extractable therefrom via line 234, that comprises liquid hydrocarbons, including, but not necessarily limited to, dimers and oligomers of the primarily olefin feed to second reactor R2′, mono- and multiply-substituted aromatic compounds, isomerized olefins, saturated hydrocarbons, naphthenes, and/or saturated and unsaturated cyclic hydrocarbons. Third separator S3′ may be fluidly connected via line 236, a first compressor C1′, and line 228 with an inlet of second reactor R2′, whereby unreacted olefins removed from third separator S3′ via line 236 may be compressed and reintroduced into second reactor R2′ for further processing. Compressor C1′ may be configured to pressurize the material removed via line 236 to a pressure in the range of from about 2100 kPa to about 4200 kPa. In embodiments, third separator S3′ is configured to provide a liquid product, removable therefrom via line 234, that comprises at least 95, 96, 97, 98, 99, or substantially all hydrocarbons. In embodiments, third separator S3′ is configured to provide a liquid product, removable therefrom via line 234, that comprises at least 20 volume percent oligomers, at least 50 volume percent oligomers, or at least 75 volume percent oligomers. In embodiments, third separator S3′ is configured to provide a liquid product, removable therefrom via line 234, that comprises at most 25 volume percent of ethylene and/or propylene, at most 10 volume percent of ethylene and/or propylene, or at most 2 volume percent of ethylene and/or propylene.

System 200 further comprises third reactor R3′ and fourth reactor R4′. Third reactor R3′ will be described further hereinbelow. Fourth reactor R4′ is a secondary oligomerization reactor fluidly connected with third separator S3′ via line 234. Fourth reactor R4′ is configured for and/or contains catalyst operable for converting the olefin dimers and oligomers of the olefins that are produced in reactor R2′ to longer chain olefins. Fourth reactor R4′ may be any reactor known in the art to be suitable for the conversion of dimers and oligomers of olefins to longer chain olefins. For example, by way of non-limiting example, fourth reactor R4′ may contain, in embodiments, a zeolite catalyst. In embodiments, fourth reactor R4′ contains ZSM-5 catalyst. Although the primary function of fourth reactor R4′ is to increase the chain length of olefins, a portion of the reactive olefins may be converted to aromatic compounds, a portion may be isomerized to isoalkenes, a portion may be converted to cyclic compounds and/or a portion may undergo cracking and/or disproportioning to smaller saturated and/or unsaturated compounds.

In embodiments, fourth reactor R4′ is operable to provide primarily C1-C30 hydrocarbons. Fourth reactor R4′ may be operable to provide a product, removable therefrom via line 244, comprising 70 volume percent product characterizable as naphtha and/or gasoline, 20 volume percent product characterizable as middle distillate (jet fuel and/or diesel fuel), and/or 10 volume percent product characterizable as heavy oil; 10 volume percent product characterizable as naphtha and/or gasoline, 15 volume percent product characterizable as middle distillate (jet fuel and/or diesel fuel), and/or 75 volume percent product characterizable as heavy oil; or 15 volume percent product characterizable as naphtha and/or gasoline, 70 volume percent product characterizable as middle distillate (jet fuel and/or diesel), and/or 15 volume percent product characterizable as heavy oil.

The operating conditions of fourth reactor R4′ will depend on the selection of catalyst and the composition of the feed stream(s) introduced thereto via line 234, 256 and/or 258. Thus, fourth reactor R4′ may be operated as known in the art to provide a stream comprising heavier olefins. In embodiments, fourth reactor R4′ is operated at a temperature in the range of from about 100° C. to about 400° C., from about 240° C. to about 350° C., or from about 275° C. to about 325° C.

In embodiments, fourth reactor R4′ is operated at a pressure in the range of from about 25 psia (172.4 kPa) to about 1000 psia (6894.8 kPa) from about 1034 kPa to about 4140 kPa, or from about 1380 kPa to about 2760 kPa.

In embodiments, second reactor R4′ is operated at a weight hourly space velocity (WHSV) in the range of from about 0.001 to about 10 hr⁻¹, from about 0.1 to about 2.0 hr⁻¹, or from about 0.25 to about 1.0 hr⁻¹.

System 200 further comprises fourth separator S4′. Fourth separator S4′ is fluidly connected with fourth reactor R4′ via line 244, whereby product of reactor R4′ may be introduced into fourth separator S4′. In embodiments, fourth separator S4′ comprises any separator suitable to separate a feed introduced thereto via line 244 into three or more products, including at least a first product comprising light olefinic vapor, a second product comprising one or more olefins selected from the group consisting of C2 through C20 olefins, and a third product comprising naphtha, middle distillate, or heavier oil. Fourth separator S4′ is fluidly connected with an outlet line 254 configured for removing a first product comprising light olefinic vapor, an outlet line 251 for removing a second product comprising one or more olefins selected from the group consisting of C2 through C20 olefins, and an outlet line 246 configured for removing a third product comprising naphtha, middle distillate, or heavier oil.

Fourth separator S4′ may be configured to provide a first product, extractable via line 254, comprising light olefinic vapor. In embodiments, fourth separator S4′ is configured to provide a first product, extractable via line 254, comprising at least about 20 weight percent olefins, at least about 50 weight percent olefins, or at least about 70 weight percent olefins. Fourth separator S4′ may be configured to provide a second product, extractable via line 251, comprising at least about 20 weight percent olefins selected from the group consisting of C2 through C20, at least about 50 weight percent olefins selected from the group consisting of C2 through C20, or at least about 90 weight percent olefins selected from the group consisting of C2 through C20. Fourth separator S4′ may be configured to provide a second product, extractable via line 246, comprising at least about 20 weight percent middle distillate (e.g. kerosene), at least about 50 weight percent middle distillate (e.g. kerosene), or at least about 90 weight percent middle distillate (e.g. kerosene). Fourth separator S4′ may be configured to provide a second product, extractable via line 252, comprising at least about 20 weight percent olefins selected from the group consisting of C2 through C20, at least about 50 weight percent olefins selected from the group consisting of C2 through C20, or at least about 90 weight percent olefins selected from the group consisting of C2 through C20.

Fourth separator S4′ may be configured to provide a third product, extractable via line 246, comprising at least about 20 weight percent middle distillate (e.g. kerosene), at least about 50 weight percent middle distillate (e.g. kerosene), or at least about 90 weight percent middle distillate (e.g. kerosene). The olefin composition of the product can be as high as 100%, but reactive disproportionation of olefins in R4′ into aromatics and alkanes may reduce the number of olefinic molecules via rearrangement into other chemical moieties. Fuel products can beneficially contain aromatics, isoalkanes, normal paraffins and/or other non-olefinic hydrocarbons. Therefore, a pure olefinic stream may be less desirable than a product stream that comprises such a mixture of hydrocarbons.

System 200 may further comprise second compressor C2′ located downstream of fourth separator S4′, whereby all or a portion of the first product comprising olefins removed from fourth separator S4′ may be recycled. Compressor C2′ may be configured to provide a compressed stream via line 259 to second reactor R2′, a compressed stream via line 256 to fourth reactor R4′, or both.

Optionally, a portion of the second product, comprising one or more olefins selected from the group consisting of C2 through C20 olefins, removable from fourth separator S4′ via line 251, may be may be returned to fourth reactor R4′ via, for example, line 248, pump P1′ and line 258. Such recycle of a portion of the third product to the fourth reactor R4′ may enable improved processing and/or higher conversion of olefins to olefin dimers and oligomers and/or create oligomers of higher molecular weight.

System 200 further comprises third reactor R3′, which is a hydrogenation reactor configured for and/or containing catalyst operable to catalyze the saturation of unsaturated compounds (i.e. olefins and other unsaturated compounds) introduced thereto. Third reactor R3′ is fluidly connected with fourth separator S4′ via line 246. Third reactor R3′ may be any reactor known in the art to be suitable for the saturation of hydrocarbons. For example, by way of non-limiting example, third reactor R3′ may contain Pd impregnated ZSM-5 catalyst or Ni/Mo on alumina catalyst. A line 232 is configured to introduce hydrogen into third reactor R3′. Third reactor R3′ is operable at conditions of WHSV, temperature and pressure effective for the conversion of at least a portion of the unsaturated components (e.g. olefinic content) of the feed introduced thereto via line 246 into more saturated or substantially entirely saturated product. Third reactor R3′ is configured to provide a product, extractable therefrom via line 238, comprising saturated hydrocarbons, aromatic hydrocarbons and/or olefinic hydrocarbons, wherein the degree of unsaturation (or the olefinic content) of the product extracted via line 238 is less than or equal to the degree of unsaturation (or the olefinic content) of the feed introduced thereto via line 246. In embodiments, system 200 further comprises a line 242 configured for recycle of at least a portion of the product removed from third reactor R3′ via line 238 to fourth reactor R4′. The product recycled in this manner may serve as a substantially non-reactive component and thus moderate one or more fourth reactor R4′ operating condition, such as, but not limited to, temperature, pressure and WHSV. In such embodiments, a line 240 may be configured for the removal of the non-recycled product from system 200.

In this manner, the third product from fourth separator S4′ may be conveyed to third reactor R3′ wherein it may be reacted with hydrogen introduced thereto via line 232 to form product which is removable therefrom via line 238. Third reactor R3′ is configured to provide a product, removable therefrom via line 238, that comprises an increased portion of saturated or more highly saturated hydrocarbons due to hydrogenation of reactive olefins and/or other unsaturated compounds.

In embodiments, third reactor R3′ is operated at a temperature in the range of from about 150° C. to about 350° C., from about 200° C. to about 300° C., or from about 250° C. to about 270° C. In embodiments, third reactor R3′ is operated at a pressure in the range of from about 2100 kPa to about 6900 kPa, from about 2700 kPa to about 5500 kPa, or from about 3400 kPa to about 4200 kPa.

System 200 is configured to provide a product, removable therefrom via lines 252 and 240, comprising liquid hydrocarbons. In embodiments, system 200 is operable to provide a product, removable via line 252, comprising olefinic naphtha and lighter olefinic and saturated hydrocarbons. In embodiments, system 200 is operable to provide a product, removable via line 240, comprising naphtha, gasoline, middle distillate and/or heavier oil.

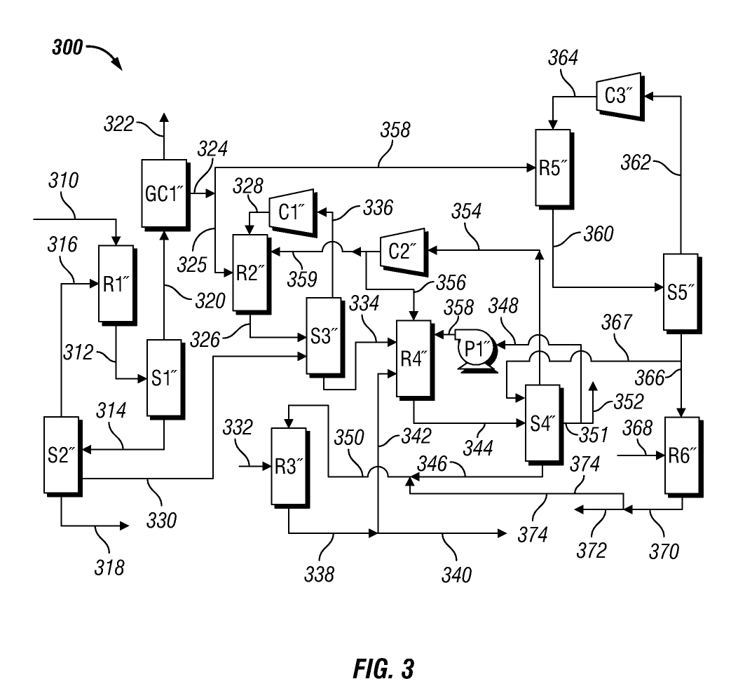

System Comprising Three Oligomerization Reactors. As mentioned hereinabove, a system according to this disclosure may comprise a plurality of oligomerization reactors. The system may also comprise a plurality of hydrogenation reactors. For example, another system for the production of liquid fuel from non-hydrocarbon components according to this disclosure will now be described with reference to FIG. 3, which depicts a system 300 comprising reactors R1″, R2″, R3″, R4″, R5″ and R6″. In this embodiment, first reactor R1″ is an olefin production reactor, second reactor R2″ is a first oligomerization reactor, third reactor R3″ is a hydrogenation reactor, fourth reactor R4″ is a second oligomerization reactor, fifth reactor R5″ is a third oligomerization reactor and sixth reactor R6″ is a second hydrogenation reactor. System 300 further comprises separators S1″, S2″, S3″, S4″ and S5″, compressors C1″, CT″ and C3″, gas cleanup apparatus GC1″, and pump P1″. Each of these components will be described in more detail hereinbelow. Thus, system 300 comprises essentially the same components as system 200 of the embodiment FIG. 2, with the addition of reactors R5″ and R6″, separator S5″, and compressor C3″. The various components may, however, be operable at different conditions and/or with different catalysts from those described with reference to FIG. 2.

System 300 comprises first reactor R1″, which is an olefin production reactor configured for converting oxygen-containing compounds introduced thereto via line 310 primarily to olefins. First reactor R1″ may be configured to convert, primarily to olefins, a feed comprising one or more oxygen-containing components selected from C1 and higher alcohols, water, C2 and similar oxygenates including, but not limited to, acids, ethers, epoxides, aldehyes and other oxygen containing compounds. First reactor R1″ contains a catalyst that is capable of catalyzing the conversion of oxygen containing compounds (i.e. ‘oxygenates’) primarily to olefins. Any suitable catalyst known in the art may be utilized. In embodiments, first reactor R1″ comprises an aluminum oxide catalyst. First reactor R1″ may be configured to convert, primarily to olefins, a feed comprising components that are liquid either in pure form or in mixture at conditions of higher pressure and/or lower temperature than the operating conditions of first reactor R1″. First reactor R1″ may be operable to provide a product, that is removable therefrom via line 312, comprising at least 50% of the available carbon as olefin product, at least 90% of the available feed carbon as olefin product, or at least 95% of the available feed carbon as olefin product.

First reactor R1″ may be configured for the production of C2 and/or C3 olefins via one or more process selected from: dehydration of alcohols including, without limitation, methanol, ethanol, propanol, butanol and/or heavier mono-alcohols, polyols, oxygenates, aldehydes, ketones and ethers; hydrogenation of acetylenic compounds formed by known methods including, without limitation, pyrolysis of natural gas and other hydrocarbons, hydration of calcium carbide, dehydrohalogenation of monochlorinated and polychlorinated hydrocarbons, and other known methods of producing compounds containing olefin functionality from C2 through C3.

The operating conditions of first reactor R1″ will depend on the selection of catalyst and the composition of the feed stream introduced thereto via line 310. Thus, first reactor R1″ may be operated as known in the art to provide a stream comprising primarily olefins. In embodiments, first reactor R1″ is operated at an operating temperature in the range of from about 50° C. to about 400° C., from about 100° C. to about 375° C., or from about 250° C. to about 350° C. In embodiments, first reactor R1″ is operated at an operating pressure in the range of from about 15 psia to about 800 psia (103 kPa to 5500 kPa), from 50 psia to about 400 psia (340 kPa to 2800 kPa), or from about 250 psia to about 300 psia (1700 kPa to 2100 kPa).

System 300 further comprises first separator 1″. First separator 1″ may be a phase separator configured to separate a liquid phase from a gas phase. First separator S1″ may be any suitable phase separator known in the art to be suitable for separating the primarily olefin product of first reactor R1″ into desired liquid and gas phases. First separator S1″ is fluidly connected via line 312 to first reactor R1″, whereby a stream comprising primarily olefins formed within first reactor R1″ may be introduced into first separator S1″. First reactor R1″ may be configured to provide a product, introducible into first separator S1″ via line 312, that comprises primarily olefins, but may further comprise one or more components selected from ethers, acetaldehyde, aldehydes, water, carbon monoxide, carbon dioxide and C1 through C16 hydrocarbons. In embodiments, phase separator S1″ is configured to separate a liquid phase comprising primarily water, heavier oxygenates and C4+ hydrocarbons, removable therefrom via line 314, from a gas phase, which is removable therefrom via a line 320. First separator 1″ may be configured to provide a vapor phase, removable therefrom via line 320, that comprises primarily olefins. In embodiments, first separator S1″ is configured to provide a vapor phase comprising at least about 20% of the available olefins, at least about 75% of the available olefins, or at least about 90% of the available olefins. In embodiments, first separator S1″ is configured to provide a vapor phase comprising at least about 20 volume percent of the ethylene introduced thereto, at least about 75 volume percent of the ethylene introduced thereto, or at least about 90 volume percent of the ethylene introduced thereto.

System 300 further comprises second separator S2″, configured to separate the produced liquid from first separator S1″ into a water phase, a liquid hydrocarbon phase, and a light vapor stream. Second separator S2″ is fluidly connected via line 314 with first separator S1″. Second separator S2″ may be any separator known in the art to be suitable for separating a liquid hydrocarbon stream from a light vapor stream and a water phase. In embodiments, second separator S2″ is selected from partial flash drums, single stage flash drums, multiple stage distillation towers and similar devices operable to separate the liquid removed from first separator S1″ into desired water, liquid hydrocarbon and light vapor streams. A line 318 is configured for the removal of a water phase from second separator S2″. A line 330 is configured for the removal of a liquid hydrocarbon phase from second separator S2″. In embodiments, second separator S2″ is operable to provide a liquid hydrocarbon phase, removable therefrom via line 330, that comprises at least 50 volume percent hydrocarbons, at least 80 volume percent hydrocarbons, or at least 95 volume percent hydrocarbons. In embodiments, line 330 fluidly connects second separator S2″ with a third separator S3″ (discussed further hereinbelow), whereby hydrocarbons in the liquid hydrocarbon phase separated from separator S2″ may be introduced into reactor R2″ and/or reactor R4″ (discussed further hereinbelow) for further processing.

A line 316 is configured for removal of a light vapor stream from second separator S2″. Second separator S2″ may be configured to provide a light vapor stream comprising oxygenates. In embodiments, second separator S2″ is configured to provide a vapor phase, removable via line 316, that comprises at least 5 volume percent oxygenates, at least 10 volume percent oxygenates, or at least 50 volume percent oxygenates. In such embodiments, line 316 may fluidly connect second separator S2″ with first reactor R1″, whereby oxygenates in the light vapor stream may be returned to first reactor R1″ for reprocessing.

System 300 further comprises gas cleanup apparatus GC1″, configured for removal of non-olefin gas species from the vapor phase removed from phase separator S1″ via line 320. A line 322 is configured for removal of primarily non-olefin components from GC1″, and a line 324 is configured for removal of a purified olefin stream from GC″.

Gas cleanup apparatus GC1″ may be configured for substantially complete removal of non-olefins from a vapor introduced thereto. In embodiments, GC1″ is configured to provide a product, removable therefrom via line 324, that comprises less than about 5 volume percent non-olefin components, less than about 0.5 volume percent non-olefin components, or less than about 0.01 volume percent non-olefin components.

System 300 further comprises second reactor R2″, which is an oligomerization reactor configured for converting olefins primarily to oligomers, while minor amounts of aromatics and/or other hydrocarbons may also be produced. Second reactor R2″ is fluidly connected with gas cleanup apparatus GC1″ via lines 324 and 325, whereby a portion of the purified olefin stream extracted from gas cleanup apparatus GC1″ may be introduced into second reactor R2″. Second reactor R2″ may be any suitable reactor known in the art to be suitable for, and may contain any catalyst known in the art to be operable to catalyze, the conversion of olefins to oligomers. For example, in embodiments, second reactor R2″ contains a zeolite catalyst. In embodiments, second reactor R2″ comprises nickel activated or nickel imbibed ZSM-5 catalyst. In embodiments, second reactor R2″ comprises nickel imbibed ZSM-5 catalyst comprising from about 0.01 weight percent to about 15 weight percent nickel, from about 1 weight percent to about 10 weight percent nickel, or from about 2 weight percent to about 8 weight percent nickel. Second reactor R2″ may be operable to provide a product comprising components selected from dimers and oligomers of the olefins introduced thereto, mono- and multiply substituted aromatic compounds, isomerized olefins, saturated hydrocarbons, naphthenes, saturated and unsaturated cyclic hydrocarbons, and/or unreacted feed components. In embodiments, second reactor R2″ is configured to provide a product, extractable therefrom via line 326, comprising ethylene, propylene, butylenes, pentenes, hexenes and/or higher mono-olefins. In embodiments, second reactor R2″ is configured to provide a product, extractable therefrom via line 326, comprising at least 20, 30, 40, 50 or 60 volume percent butylenes and/or heavier olefins. In embodiments, second reactor R2″ is configured to provide a product that comprises primarily butene or primarily butene and hexene. In embodiments, second reactor R2″ is configured to provide a product that comprises primarily components selected from the group consisting of butenes, hexenes, octenes, decenes, and heavier mono-olefins, oligomers, naphthenes, and aromatic compounds. In embodiments, second reactor R2″ is configured to provide a product, extractable therefrom via line 326, that comprises at least 20% volume percent butene or butene and hexene, at least 70% volume percent butene or butene and hexene, or at least 90% volume percent butene or butene and hexene.

The operating conditions of second reactor R2″ will depend on the selection of catalyst and the composition of the feed stream introduced thereto via lines 324 and 325. Thus, second reactor R2″ may be operated as known in the art to provide a stream comprising primarily dimers of the olefins in the feed thereto (e.g. oligomers). In embodiments, second reactor R2″ is operated at a temperature in the range of from about from about 80° C. to about 400° C., from about 100° C. to about 280° C., or from about 170° C. to about 240° C.

In embodiments, second reactor R2″ is operated at a pressure in the range of from about 200 psia (1380 kPa) to about 600 psia (4140 kPa), from about 1650 kPa to about 2400 kPa, or from about 1790 kPa to about 2210 kPa. In embodiments, the operating pressure of first reactor R1″ is greater than the operating pressure of second reactor R2″. In such embodiments, the system may not comprise a compressor for conveying the gaseous products from reactor R1″ to reactor R2″. That is, in embodiments, dehydration is performed in first reactor R1″ at a pressure above the oligomerization pressure of second reactor R2″.

In embodiments, second reactor R2″ is operated at a weight hourly space velocity (WHSV) in the range of from about 0.001 to about 10 hr⁻¹, from about 0.1 to about 2.0 hr⁻¹, or from about 0.25 to about 1.0 hr⁻¹.

System 300 further comprises a fifth reactor R5″, which is an oligomerization reactor configured for converting olefins to larger olefins, aromatics and/or other hydrocarbons by the processes of oligomerization and aromatization. Fifth reactor R5″ is fluidly connected with gas cleanup apparatus GC1″ via lines 324 and 358, whereby a portion of the purified olefin stream extractable from gas cleanup apparatus GC1″ via line 324 may be introduced into fifth reactor R5″.

Fifth reactor R5″ may be any suitable reactor known in the art to be suitable for, and may contain any catalyst known in the art to be operable to catalyze, the conversion of olefins to larger olefins, aromatics and/or other hydrocarbons. For example, in embodiments, fifth reactor R5″ contains a zeolite catalyst. In embodiments, fifth reactor R5″ comprises ZSM-5 catalyst. Fifth reactor R5″ may be operable to provide a product comprising one or more components selected from oligomers of the olefins introduced thereto via line 358, mono and multiply substituted aromatic compounds, isomerized olefins, saturated hydrocarbons, naphthenes, saturated and unsaturated cyclic hydrocarbons, and unreacted feed components. In embodiments, fifth reactor R5″ is configured to provide a product, extractable therefrom via line 360, comprising at least or about 50% of the available carbon as olefin product, at least 90% of the available feed carbon as olefin product, or at least 95% of the available feed carbon as olefin product. In embodiments, fifth reactor R5″ is configured to provide a product, extractable therefrom via line 360, comprising from about 4 volume percent aromatics to about 85 volume percent aromatics.

The operating conditions of fifth reactor R5″ will depend on the selection of catalyst and the composition of the feed stream introduced thereto via line(s) 358 and/or 364. Thus, fifth reactor R5″ may be operated as known in the art to provide a stream comprising primarily oligomers of the olefins introduced thereto via line 358. In embodiments, fifth reactor R5″ operates under the same or similar conditions as described herein for reactor R2. In embodiments, fifth reactor R5″ is operated at an operating temperature in the range of from about 50° C. to about 450° C., from about 100° C. to about 400° C., or from about 250° C. to about 350° C. In embodiments, fifth reactor R5″ is operated at an operating pressure in the range of from about 15 psia to about 800 psia (103 kPa to 5500 kPa), from about 50 psia to about 400 psia (340 kPa to 2800 kPa), or from about 250 psia to about 300 psia (1700 kPa to 2100 kPa).

System 300 further comprises fifth separator S5″, configured for separating a liquid hydrocarbon stream from a vapor phase comprising primarily unreacted olefinic compounds. Fifth separator S5″ may be operable under the same or similar conditions as described hereinabove for separator S2. Fifth separator S5″ is fluidly connected with fifth reactor R5″ via line 360, whereby product from fifth reactor R5″ may be introduced into fifth separator S5″. Fifth separator S5″ may be any suitable phase separator known in the art to separate a liquid hydrocarbon stream from unreacted, gaseous olefins. A line 366 is configured for the removal of liquid hydrocarbons from fifth separator S5″, while a line 362 is configured for removal therefrom of a gaseous/vapor phase comprising unreacted olefins. Fifth separator S5″ may be configured to provide a liquid, extractable therefrom via line 366, that comprises liquid hydrocarbons, including, but not necessarily limited to, oligomers of the primarily olefin feed to reactor R5″, mono- and multiply-substituted aromatic compounds, isomerized olefins, saturated hydrocarbons, naphthenes, and/or saturated and unsaturated cyclic hydrocarbons. Fifth separator S5″ may be fluidly connected via line 362, a third compressor C3″, and line 364 with an inlet of fifth reactor R5″, whereby unreacted olefins removed from fifth separator S5″ via line 362 may be compressed and reintroduced into fifth reactor R5″ for further processing. Third compressor C3″ may be configured to pressurize materials introduced thereto to a pressure in the range of from about 1700 kPa to about 5500 kPa. In embodiments, fifth separator S5″ is configured to provide a liquid product, removable therefrom via line 366, that comprises at least 20% of the available olefins, at least 75% of the available olefins, or at least 90% of the available olefins introduced thereto. In embodiments, fifth separator S5″ is configured to provide a liquid product, removable therefrom via line 366, that comprises at least 20 volume percent of the unsaturated hydrocarbons introduced thereto, at least 75 volume percent of the unsaturated hydrocarbons introduced thereto, or at least 90 volume percent of the unsaturated hydrocarbons introduced thereto.

At least a portion of the liquid hydrocarbons removed from separator S5″ may also be processed jointly with the products of reactor R4″ by introducing them to separator S4″ via line 367.

System 300 further comprises sixth reactor R6″, which is a hydrogenation reactor configured for and/or containing catalyst operable to catalyze the saturation of olefins introduced thereto, thus increasing the degree of saturation of a feed introduced thereto. Sixth reactor R6″ is fluidly connected with fifth separator S5″ via line 366. Sixth reactor R6″ may be any reactor known in the art to be suitable for the saturation of hydrocarbons. For example, by way of non-limiting example, sixth reactor R6″ may contain Pd impregnated ZSM-5 catalyst or Ni/Mo on alumina catalyst. A line 368 is configured to introduce hydrogen into sixth reactor R6″. Sixth reactor R6″ is operable at conditions of WHSV, temperature and pressure effective for the conversion of at least a portion of the unsaturated (e.g. olefinic) content of the feed introduced thereto via line 366 into more saturated or substantially entirely saturated product. Sixth reactor R6″ is configured to provide a product, extractable therefrom via line 370, comprising saturated hydrocarbons, aromatic hydrocarbons and/or olefinic hydrocarbons, wherein the degree of unsaturation (or the olefinic content) of the product extracted via line 370 is less than or equal to the degree of unsaturation (or the olefinic content) of the feed introduced thereto via line 366. In embodiments, system 300 further comprises a line 374 configured for introduction of at least a portion of the product removed from sixth reactor R6″ via line 370 to third reactor R3″, whereby the olefinic content of the material introduced into third reactor R3″ via lines 374 and/or 350 may be further reduced. In such embodiments, a line 372 may be configured for the removal of the non-recycled product (e.g. for storage and/or etc.) from system 300.

In embodiments, sixth reactor R6″ is operated in the same or similar operating ranges as described hereinabove for reactor R3. In embodiments, sixth reactor R6″ is operated at a temperature in the range of from about 150° C. to about 350° C., from about 200° C. to about 300° C., or from about 250° C. to about 270° C. In embodiments, sixth reactor R6″ is operated at a pressure in the range of from about 2100 kPa to about 6900 kPa, from about 2700 kPa to about 5500 kPa, or from about 3400 kPa to about 4200 kPa.

System 300 further comprises third separator S3″, configured for separating a liquid hydrocarbon stream from a vapor phase comprising primarily unreacted olefinic compounds. Third separator S3″ is fluidly connected with second reactor R2″ via line 326, whereby product from second reactor R2″ may be introduced into third separator S3″. Third separator S3″ may be any suitable phase separator known in the art to separate a liquid hydrocarbon stream from unreacted, gaseous olefins. A line 334 is configured for the removal of liquid hydrocarbons from third separator S3″, while a line 336 is configured for removal therefrom of a gaseous/vapor phase comprising unreacted olefins. Third separator S3″ may be configured to provide a liquid, extractable via line 334, that comprises liquid hydrocarbons, including, but not necessarily limited to, one or more component selected from dimers and oligomers of the primarily olefin feed to reactor R2″, mono- and multiply-substituted aromatic compounds, isomerized olefins, saturated hydrocarbons, naphthenes, and saturated and unsaturated cyclic hydrocarbons. Third separator S3″ may be fluidly connected via line 336, a first compressor C1″, and line 328 with an inlet of second reactor R2″, whereby unreacted olefins removed from third separator S3″ via line 336 may be compressed and reintroduced into second reactor R2″ for further processing. Compressor C1″ may be configured to pressurize materials introduced thereto to a pressure in the range of from about 1380 kPa to about 4140 kPa. In embodiments, third separator S3″ is operable in the same or similar operating ranges as separator S3′. In embodiments, third separator S3″ is configured to provide a liquid product, removable therefrom via line 334, that comprises at least 90 volume percent hydrocarbons, at least 95 volume percent hydrocarbons, or at least 97 volume percent hydrocarbons. In embodiments, third separator S3″ is configured to provide a liquid product, removable therefrom via line 334, that comprises at least 20 volume percent oligomers, at least 50 volume percent oligomers, or at least 75 volume percent oligomers

System 300 further comprises third reactor R3″ and fourth reactor R4″. Third reactor R3″ will be described further hereinbelow. Fourth reactor R4″ is fluidly connected with third separator S3″ via line 334. Fourth reactor R4″ is an oligomerization reactor configured for and/or contains catalyst operable for converting the olefin dimers and oligomers of the olefins that are produced in reactor R2″ to longer chain olefins. Fourth reactor R4″ may be any reactor known in the art to be suitable for the conversion of dimers and oligomers of olefins to longer chain olefins. In embodiments, fourth reactor R4″ contains a solid acid catalyst such as a silica aluminate, for example ZSM-5. For example, by way of non-limiting example, fourth reactor R4″ may contain a zeolite catalyst. In embodiments, fourth reactor R4″ contains ZSM-5 catalyst. In embodiments, fourth reactor R4″ contains nickel activated ZSM-5 catalyst. Although the primary function of fourth reactor R4″ is to increase the chain length of olefins, a portion of the reactive olefins may be converted to aromatic compounds, a portion may be isomerized to isoalkenes, a portion may be converted to cyclic compounds and/or a portion may undergo cracking and/or disproportioning to smaller saturated and/or unsaturated compounds.

In embodiments, fourth reactor R4″ is operable to provide primarily C1-C30 hydrocarbons. Fourth reactor R4″ may be operable to provide a product, removable therefrom via line 344, comprising about 70 volume product characterizable as naphtha and/or gasoline, about 20 volume percent product characterizable as middle distillates (jet fuel and/or diesel), and/or about 10 volume percent product characterizable as heavy oil; comprising about 10 volume percent product characterizable as naphtha and/or gasoline, 15 volume percent product characterizable as middle distillates (jet fuel and/or diesel), and/or 75 volume percent product characterizable as heavier oil; or about 15 volume percent product characterizable as naphtha and gasoline, about 70 volume percent product characterizable as middle distillates, and/or 15 volume percent product characterizable as heavy oil.

The operating conditions of fourth reactor R4″ will depend on the selection of catalyst and the composition of the feed stream(s) introduced thereto via line 334, 356 and/or 358. Thus, fourth reactor R4″ may be operated as known in the art to provide a stream comprising heavier olefins. In embodiments, fourth reactor R4″ is operated at a temperature in the range of from about 100° C. to about 400° C., from about 150° C. to about 350° C., or from about 175° C. to about 300° C.

In embodiments, fourth reactor R4″ is operated at a pressure in the range of from about 25 psia (172.4 kPa) to about 1000 psia (6894.8 kPa), from about 1034 kPa to about 4140 kPa, or from about 1380 kPa to about 2760 kPa.

In embodiments, fourth reactor R4″ is operated at a weight hourly space velocity (WHSV) in the range of from about 0.001 hr⁻¹ to about 10 hr⁻¹, from about 0.1 hr⁻¹ to about 2.0 hr⁻¹, or from about 0.25 hr⁻¹ toabout 1.0 hr⁻¹.

System 300 further comprises fourth separator S4″. Fourth separator S4″ is fluidly connected with fourth reactor R4″ via line 344, whereby product of reactor R4″ may be introduced into fourth separator S4″. In embodiments, fourth separator S4″ comprises any separator suitable to separate a feed introduced thereto via line 344 into three or more products, including at least a first product comprising olefinic vapor, a second product comprising one or more olefins selected from the group consisting of C2 through C20 olefins, and a third product comprising olefinic naphtha, middle distillates, or heavy oil. Fourth separator R4″ is fluidly connected with an outlet line 354 configured for removing a first product comprising light olefinic vapor, an outlet line 351 for removing a second product comprising one or more olefins selected from the group consisting of C2 through C20 olefins and an outlet line 346 configured for removing a third product comprising naphtha, middle distillates, and/or heavier oils.

Fourth separator S4″ may be operable in the same ranges as described hereinabove for separator S4′. Fourth separator S4″ may be configured to provide a first product, extractable via line 354, comprising light olefinic vapor. Fourth separator S4″ may be configured to provide a first product, extractable via line 354, comprising at least about 20 weight percent olefins, at least about 50 weight percent olefins, or at least about 70 weight percent olefins.

Fourth separator S4″ may be configured to provide a second product, extractable via line 351, comprising at least about 20 weight percent naphtha, at least about 50 weight percent naphtha, or at least about 90 weight percent naphtha. Fourth separator S4″ may be configured to provide a second product, extractable via line 351, comprising of at least about 40 weight percent of one or more olefins selected from the group consisting of C2 through C20 olefins, at least about 60 weight percent of one or more olefins selected from the group consisting of C2 through C20 olefins, or at least about 80 weight percent of one or more olefins selected from the group consisting of C2 through C20 olefins.

Fourth separator S4″ may be configured to provide a third product, extractable via line 346, comprising at least about 40 weight percent olefinic naphtha, middle distillate, and/or heavier oil, at least about 60 weight percent olefinic naphtha, middle distillate, and/or heavier oil, or at least about 80 weight percent olefinic naphtha, middle distillate, and/or heavier oil.

System 300 may further comprise second compressor C2″ located downstream of fourth separator S4″, whereby all or a portion of the first product comprising olefins removed from fourth separator S4″ may be recycled. Compressor C2″ may be configured to provide a compressed stream via line 359 to second reactor R2″, a compressed stream via line 356 to fourth reactor R4″, or both.

Optionally, a portion of the second product, comprising one or more olefins selected from the group consisting of C2 through C20 olefins, removable from fourth separator S4″ via line 346, may be may be returned to fourth reactor R4″ via, for example, line 348, pump P1″ and line 358. Such recycle of a portion of the second product to the fourth reactor R4″ may enable improved processing and/or higher conversion of olefins to olefin dimers and oligomers and/or create oligomers of higher molecular weight.

System 300 further comprises third reactor R3″, which is a hydrogenation reactor configured for and/or containing catalyst operable to catalyze the saturation of unsaturated components (e.g. olefins) introduced thereto. Third reactor R3″ is fluidly connected with fourth separator S4″ via line 346. Third reactor R3″ may be any reactor known in the art to be suitable for the saturation of hydrocarbons. For example, by way of non-limiting example, third reactor R3″ may contain Pd impregnated ZSM-5 catalyst or Ni/Mo on alumina catalyst. A line 332 is configured to introduce hydrogen into third reactor R3″. Third reactor R3″ is operable at conditions of WHSV, temperature and pressure effective for the conversion of at least a portion of the unsaturated components (e.g. olefinic content) of the feed introduced thereto via line 346 into more saturated or substantially entirely saturated product. Third reactor R3″ is configured to provide a product, extractable therefrom via line 338, comprising saturated hydrocarbons, aromatic hydrocarbons and/or olefinic hydrocarbons, wherein the degree of unsaturation (or the olefinic content) of the product extracted via line 338 is less than or equal to the degree of unsaturation (or the olefinic content) of the feed introduced thereto via line 346.

In embodiments, system 300 further comprises a line 342 configured for recycle of at least a portion of the product removed from third reactor R3″ via line 338 to fourth reactor R4″. The product recycled in this manner may serve as a substantially non-reactive component and thus moderate one or more fourth reactor R4″ operating condition, such as, but not limited to, temperature, pressure and WHSV. In such embodiments, a line 340 may be configured for the removal of the non-recycled product from system 300.

In this manner, the portion of third product from fourth separator S4″ that is not returned to fourth reactor R4″ as a processing aid and/or to enhance the overall process conversion may be conveyed to third reactor R3″ wherein it may be reacted with hydrogen introduced thereto via line 332 to form product which is removable from third reactor R3″ via line 338. Third reactor R3″ may be configured to provide a product, removable therefrom via line 338, that comprises an increased portion of saturated hydrocarbons due to hydrogenation of reactive olefins and/or other unsaturated compounds.

In embodiments, third reactor R3″ is operated at a temperature in the range of from about 150° C. to about 350° C., from about 200° C. to about 300° C., or from about 250° C. to about 270° C. In embodiments, third reactor R3″ is operated at a pressure in the range of from about 2100 kPa to about 6900 kPa, from about 2700 kPa to about 5500 kPa, or from about 3400 kPa to about 4200 kPa.

System 300 is configured to provide products, removable therefrom via lines 340, 352 and 372, comprising hydrocarbons. In embodiments, system 300 is operable to provide a product, removable via in line 340, that comprises naphtha, gasoline, middle distillate and/or heavier oil. In embodiments, system 300 is operable to provide a product, removable via line 352, that comprises olefinic naphtha and lighter olefinic and/or saturated hydrocarbons. In embodiments, system 300 is operable to provide a product, removable via line 372, that comprises naphtha, middle distillates, and/or heavier oils.

Although exemplary systems comprising three, four and six reactors have been described in detail herein, it is envisioned that a system of this disclosure can comprise any number of reactors, from 2 to 10 and more. For example, a system according to this disclosure may comprise 5 reactors. For example, a system as depicted in FIG. 3 but lacking reactor R6″, and thus comprising five reactors, could be utilized. In such an embodiment, for example, hydrogenation reactor R3″ could be utilized to hydrogenate the products of both reactor R5″ and R4″. The disclosed system may comprise one, two, three, four, or more oligomerization reactors. The disclosed system may comprise one, two, three, four, or more hydrogenation reactors. The disclosed system may comprise one or more olefin production reactors.

Process for the Production of Liquid Fuels from Non-Hydrocarbons. Also disclosed herein is a process for producing liquid fuels from non-hydrocarbons. The process of this disclosure comprises producing olefins, producing oligomers and other hydrocarbons from the olefins produced, and producing rearranged (e.g. more saturated) hydrocarbons from the various oligomers and other hydrocarbons produced, as discussed in detail hereinbelow. The process incorporates a plurality of reactors configured for the production of olefins, the production of oligomers and other hydrocarbons from the olefins and/or for the production of rearranged hydrocarbons from the oligomers and other hydrocarbons. Suitable systems and reactors for carrying out the process of this disclosure are described in detail hereinabove.

Process Comprising Single Oligomerization. A process for the production of liquid hydrocarbons from non-hydrocarbon components according to this disclosure will now be described with reference to FIG. 1. In this embodiment, oxygen-containing compounds are converted to primarily olefins by introduction thereof via line 110 to first reactor R1, the configuration and operation of which is described hereinabove. The feed introduced into first reactor R1 may comprise one or more oxygen-containing components selected from C1 and higher alcohols, water, C2 and similar oxygenates including, but not limited to, acids, ethers, epoxides, aldehyes and other oxygen containing compounds. Within first reactor R1, the oxygen containing compounds (i.e. ‘oxygenates’) are catalytically converted to product comprising primarily olefins. A product comprising primarily olefins is removed from first reactor R1 via line 112. In embodiments, the product removed from reactor R1 via line 112 comprises at least 50% of the available carbon as olefin product, at least 90% of the available feed carbon as olefin product, or at least 95% of the available feed carbon as olefin product. The product removed from first reactor R1 via line 112 comprises primarily olefins, but may further comprise one or more components selected from alcohols, ethers, aldehydes, such as acetaldehyde, water, carbon monoxide, carbon dioxide, hydrogen, and C1 through C16 hydrocarbons.

The primarily olefin product of first reactor R1 is introduced into first separator S1, described hereinabove, whereby the primarily olefin product of first reactor R1 is separated into desired liquid and gas phases. Phase separator S1 may be operated to separate a liquid phase comprising primarily water, heavier oxygenates and C4+ hydrocarbons, which is removed therefrom via line 114, from a gas phase, which is removed via line 120. The vapor phase removed from first separator S1 via line 120 comprises primarily olefins. In embodiments, the vapor phase removed from first separator S1 via line 120 comprises at least 20% of the available olefins, at least 75% of the available olefins, or at least 90% of the available olefins.

The liquid product of separator S1 is introduced into second separator S2, which is operated to provide a water phase, a liquid hydrocarbon phase, and a light vapor stream. A water phase is removed from second separator S2 via line 118. A liquid hydrocarbon phase is removed from second separator S2 via line 130. In embodiments, the liquid hydrocarbon phase removed from second separator S2 via line 130 comprises at least 50 volume percent hydrocarbons, at least 80 volume percent hydrocarbons, or at least 95 volume percent hydrocarbons. In embodiments, hydrocarbons in the liquid hydrocarbon phase separated from separator S2 are introduced, via, for example, line 130 and third separator S3, into second reactor R2 and/or third reactor R3 (discussed further hereinbelow) for further processing.

A light vapor stream is removed from second separator S2 via line 116. The light vapor stream may comprise oxygenates. In embodiments, the vapor phase removed from second separator S2 via line 116 comprises at least 5 volume percent oxygenates, at least 10 volume percent oxygenates, or at least 50 volume percent oxygenates. In such embodiments, oxygenates in the light vapor stream may be returned to first reactor R1 for reprocessing, via line 116.

Non-olefin species may be removed from the vapor phase extracted from phase separator S1 via line 120, for example via gas cleanup apparatus GC1. In embodiments, substantially all of the non-olefins in the vapor phase extracted from separator S1 via line 120 are removed, providing a non-olefin product extracted via line 122 and a purified olefin product extracted via line 124. In embodiments, non-olefin species are removed from the vapor phase product of first separator S1 to provide a purified olefin product comprising less than about 5 volume percent non-olefin components, less than about 0.5 volume percent non-olefin components, or less than about 0.01 volume percent non-olefin components.

The olefins in the purified olefin stream are converted into larger olefins, aromatics and/or other hydrocarbons by the processes of oligomerization and/or aromatization. The purified olefin stream may be introduced into second reactor R2 via line 124. The configuration and operation of second reactor R2 is described hereinabove. Within second reactor R2, at least a portion of the olefins is catalytically converted to provide a product comprising larger olefins, aromatics and/or other hydrocarbons. Within second reactor R2, the olefins may be converted to a product comprising one or more components selected from oligomers of the olefins introduced thereto, mono- and multiply substituted aromatic compounds, isomerized olefins, saturated hydrocarbons, naphthenes, saturated and unsaturated cyclic hydrocarbons, and unreacted feed components. In embodiments, the purified olefin product is converted, via second reactor R2, into a product, extracted therefrom via line 126, comprising about 70 volume percent product characterizable as naphtha and/or gasoline, about 20 volume percent product characterizable as middle distillate, and/or about 10 volume percent product characterizable as heavier oil. In embodiments, second reactor R2 is configured to provide a product, extracted therefrom via line 126, comprising 15 volume percent product characterizable as naphtha and/or gasoline, 70 volume percent product characterizable as middle distillate, and/or 15 volume percent product characterizable as heavier oil. In embodiments, second reactor R2 is configured to provide a product, extractable therefrom via line 126, comprising about 10 volume percent product characterizable as naphtha and/or gasoline, about 15 volume percent product characterizable as middle distillate, and/or about 75 volume percent product characterizable as heavier oil.

Unreacted olefinic compounds may be removed from the product of second reactor R2 via third separator S3. Liquid hydrocarbons may be removed from third separator S3 via line 134 and a gaseous/vapor phase comprising unreacted olefins may be removed from third separator S3 via line 136. Unreacted olefins removed from third separator S3 via line 136 may be compressed via first compressor C1 and reintroduced into second reactor R2 for further processing, for example, via line 128. In embodiments, the liquid product removed from third separator S3 via line 134 comprises at least 90 volume percent hydrocarbons, at least 95 volume percent hydrocarbons, or at least 99 volume percent hydrocarbons.

The liquid product of third separator S3 may be hydrogenated to provide a more saturated product. The liquid product of third separator S2 may be introduced into third reactor R3 via line 134, whereby the degree of saturation of the product is increased. The configuration and operation of third reactor R3 is described hereinabove. Within third reactor R3, hydrogen introduced thereto via line 132 reacts with unsaturated compounds in the feed introduced thereto via line 134, to provide a (more) saturated product. More saturated or substantially entirely saturated product is extracted from third reactor R3 via line 138.

The product of reactor R3, extracted therefrom via line 138, comprises (more) saturated hydrocarbons, aromatic hydrocarbons and/or olefinic hydrocarbons, wherein the degree of unsaturation (or the olefinic content) of the product extracted via line 138 is less than or equal to the degree of unsaturation (or the olefinic content) of the feed introduced thereto via line 134. At least a portion of the product removed from third reactor R3 via line 138 may be recycled, via line 142, to second reactor R2. Such recycle material may serve as a substantially non-reactive component, thus moderating one or more operating condition of second reactor R2 and/or third reactor R3. Non-recycled, liquid hydrocarbon product of third reactor R3 may be removed via line 140. The liquid hydrocarbon product produced via this method may comprise naphtha, middle distillate and/or heavier oil.

In embodiments, the product extracted from third reactor R3 via line 140 is separated by boiling point fractionation into one or more products selected from a naphtha fraction, a heavier oil fraction, and a middle distillate fraction. The one or more products may be quantifiably differentiated by one or more of physical density, energy density, aromaticity, boiling point, olefin content and saturate content (i.e. degree of saturation). In embodiments, system 100 is operable to produce a product comprising C1-C15 hydrocarbons. In embodiments, the product comprises primarily hydrocarbons having a boiling point in the automotive gasoline range. In embodiments, at least one product comprises between about 4 and about 90 volume percent aromatics, between about 10 and about 80 volume percent aromatics, between about 30 and about 70 volume percent aromatics, between about 20 and about 50 volume percent aromatics, between about 12 and about 20 volume percent aromatics, between about 8 and about 12 volume percent aromatics, or between about 4 and about 8 volume percent aromatics.

Process Comprising Two Oligomerizations. Another process for the production of liquid fuel from non-hydrocarbon components according to this disclosure will now be described with reference to FIG. 2. A feed comprising oxygen-containing compounds is converted into a product comprising primarily olefins by introduction thereof via line 210 into first reactor R1′. The configuration and operation of first reactor R1′ are discussed hereinabove. The feed may comprise one or more oxygen-containing compounds selected from C1 and higher alcohols, water, C2 and similar oxygenates including, but not limited to, acids, ethers, epoxides, aldehyes and other oxygen containing compounds. In embodiments, the primarily olefin product produced via first reactor R1′ comprises at least about 50% of the available carbon as olefin product, at least about 90% of the available feed carbon as olefin product, or at least about 95% of the available feed carbon as olefin product. The primarily olefin product of first reactor R1′ may further comprise one or more components selected from alcohols, ethers, aldehydes such as, but not limited to acetaldehyde, water, carbon monoxide, carbon dioxide, hydrogen, and C1 through C16 hydrocarbons.

The primarily olefin product of first reactor R1′ is introduced into first separator S1′ via line 212. The configuration and operation of first separator S1′ are described hereinabove. A liquid phase comprising primarily water, heavier oxygenates and C4+ hydrocarbons may be removed from first separator S1′ via line 214, and a gas phase comprising primarily olefins removed from first separator S1′ via line 220. In embodiments, the vapor phase removed from first separator S1′ via line 220 comprises at least about 20% of the available olefins, at least about 75% of the available olefins, or at least about 90% of the available olefins.

The liquid product extracted from first separator S1′ via line 214 may be further separated into a water phase, a liquid hydrocarbon phase, and a light vapor stream. Such separation may be performed via a second separator S2′, as described hereinabove. A water phase may be removed from second separator S2′ via line 218. A liquid hydrocarbon phase may be removed from second separator S2′ via line 230. In embodiments, the liquid hydrocarbon phase removed from second separator S2′ via line 230 comprises at least *50 volume percent hydrocarbons, at least 80 volume percent hydrocarbons, or at least 95 volume percent hydrocarbons. In embodiments, hydrocarbons in the liquid hydrocarbon phase separated from separator S2′ via line 230 may be further processed by introduction via, for example, third separator S3, into second reactor R2′ and/or fourth reactor R4′.

A light vapor stream comprising oxygenates may be extracted from second separator S2′ via line 216. In embodiments, the vapor phase removed from second separator S2′ via line 216 comprises at least 5 volume percent oxygenates, at least 10 volume percent oxygenates, or at least 50 volume percent oxygenates. In such embodiments, oxygenates in the light vapor stream may be returned to first reactor R1′ via line 216 for reprocessing.

In embodiments, non-olefins are removed from the vapor phase separated via first separator S1′. For example, the vapor phase separated from first separator SP via line 220 may be introduced into gas cleanup apparatus GCl′, whereby non-olefins may be removed. In embodiments, substantially all of the non-olefins are removed from the vapor phase extracted from first separator S1′ via line 220, providing a purified olefin stream. In embodiments, a purified olefin product is removed from gas cleanup apparatus GC1′ via line 224. In embodiments, the purified olefin product comprises less than about 5 volume percent non-olefin components, less than about 0.5 volume percent non-olefin components, or less than about 0.01 volume percent non-olefin components. Primarily non-olefin components may be removed from gas cleanup apparatus GC1′ via line 222.

The olefins within the purified olefin product extracted from gas cleanup apparatus GC1′ may be converted primarily to oligomers within second reactor R2′. The configuration and operation of second reactor R2′ are described hereinabove. Second reactor R2′ may provide a product, extractable via line 226, comprising primarily oligomers. The product of second reactor R2′ may further comprise minor amounts of aromatics and/or other hydrocarbons. In embodiments, the product of second reactor R2′ comprises one or more components selected from dimers and oligomers of the olefins introduced thereto, mono- and multiply substituted aromatic compounds, isomerized olefins, saturated hydrocarbons, naphthenes, saturated and unsaturated cyclic hydrocarbons, and unreacted feed components. In embodiments, the product extracted from second reactor R2′ comprises primarily butenes and/or hexenes. Depending on economics, a portion of the product of second reactor R2′ (e.g. butene-containing product) may be separated for sale (e.g. for example, for the production of rubber). In embodiments, the product extracted from second reactor R2′ via line 226 comprises greater than or about 20 volume percent C4+ alkenes. In embodiments, the product extracted from second reactor R2′ via line 226 comprises greater than or about 90 volume percent C4+ alkenes. In embodiments, second reactor R2′ is configured to provide a product that comprises primarily butene or primarily butene and hexene. In embodiments, second reactor R2′ is configured to provide a product that comprises primarily components selected from the group consisting of butenes, hexenes, octenes, decenes, and heavier mono-olefins, paraffins, naphthenes, and aromatic compounds. In embodiments, the product extracted from second reactor R2′ via line 226 comprises ethylene, propylene, butylenes, pentenes, hexenes and/or higher mono-olefins. In embodiments, the product extracted from second reactor R2′ via line 226 comprises at least 20 volume percent butylenes and/or heavier olefins; at least 20 volume percent butene or butene and hexene; at least 40 volume percent butene or butene and hexene; or at least 60 volume percent butene or butene and hexene.

Unreacted olefinic compounds may be removed from the product of second reactor R2′ via a third separator S3′. Suitable configurations and operating parameters for third separator S3′ are provided hereinabove. Within third separator S3′, a liquid hydrocarbon stream may be separated from a vapor phase comprising primarily unreacted olefinic compounds. Liquid hydrocarbons may be removed from third separator S3′ via line 234, and a gaseous/vapor phase comprising unreacted olefins may be removed from third separator S3′ via line 236. In embodiments, the liquid extracted via line 234 comprises liquid hydrocarbons, including, but not necessarily limited to, one or more component selected from dimers and oligomers of the primarily olefin feed to reactor R2′, mono- and multiply-substituted aromatic compounds, isomerized olefins, saturated hydrocarbons, naphthenes, and saturated and unsaturated cyclic hydrocarbons. Unreacted olefins removed from third separator S3′ via line 236 may be compressed via first compressor C1′ and reintroduced via line 228 into second reactor R2′ for further processing. In embodiments, the liquid product removed from third separator S3′ via line 234 comprises at least 90 volume percent hydrocarbons, at least 95 volume percent hydrocarbons, or at least 98 volume percent hydrocarbons. In embodiments, the liquid product removed from third separator S3′ via line 234 comprises at least 20 volume percent oligomers, at least 50 volume percent oligomers, or at least 75 volume percent oligomers.

At least a portion of the olefin dimers and oligomers of the olefins that are produced in reactor R2′ may be converted to longer chain olefins. For example, in embodiments, the liquid product removed from third separator S3′ via line 234 may be introduced into fourth reactor R4′. Suitable configurations and operating conditions for fourth reactor R4 are provided hereinabove. Within fourth reactor R4′, the average chain length of the olefins fed thereto is increased. Additionally, a portion of the reactive olefins introduced into fourth reactor R4′ may be converted to aromatic compounds, a portion may be isomerized to isoalkenes, a portion may be converted to cyclic compounds and/or a portion may undergo cracking and/or disproportioning to smaller saturated and/or unsaturated compounds.

In embodiments, the product of fourth reactor R4′ comprises higher oligomers comprising from C6 to C30+ hydrocarbons. In embodiments, the product of fourth reactor R4′ comprises light olefinic vapor, olefinic naphtha, middle distillate, heavier oil, waxes, kerosene, heavier oil and lube oils, or olefinic gasoline, kerosene and heavier oil.

The product of fourth reactor R4′ may be separated into a variety of products. For example, in embodiments, the product of fourth reactor R4′ is separated into three or more products via fourth separator S4′. A first product comprising olefinic vapor, a second product comprising one or more olefins selected from the group consisting of C2 through, and a third product C20 comprising, naphtha, middle distillate, or heavier oil olefins may be separated from the feed introduced to fourth separator S4′ via line 244.

A first product may be extracted from fourth separator S4′ via line 254, the first product comprising olefinic vapor. The vapor product of S4′ can be anything that is lighter than the product of 246. The first product extracted from fourth separator S4′ via line 254 may comprise at least about 20 weight percent olefins, at least about 50 weight percent olefins, or at least about 70 weight percent olefins. This stream may also contain paraffins, hydrogen, and/or carbon oxides.

A second product may be extracted from fourth separator S4′ via line 250, the second product comprising at least about 40 weight percent of one or more olefins selected from the group consisting of C2 through C20 olefins, at least about 60 weight percent of one or more olefins selected from the group consisting of C2 through C20 olefins, or at least about 80 weight percent of one or more olefins selected from the group consisting of C2 through C20 olefins.

A third product may be extracted from fourth separator S4′ via line 246, the third product comprising at least about 20 weight percent naphtha, middle distillate, and/or heavier oil, at least about 50 weight percent naphtha, middle distillate, and/or heavier oil, or at least about 90 weight percent naphtha, middle distillate, and/or heavier oil.

All or a portion of the first product comprising olefins removed from fourth separator S4′ may be recycled via second compressor C2′ and line 259 to second reactor R2′, via second compressor C2′ and line 256 to fourth reactor R4′, or both.

Optionally, a portion of the second product, comprising one or more olefins selected from the group consisting of C2 through C20 olefins, removed from fourth separator S4′ via line 251 may be may be returned to fourth reactor R4′ via, for example, line 248, pump P1′ and line 258. Such recycle of a portion of the third product to the fourth reactor R4′ may enable improved processing and/or higher conversion of olefins to olefin dimers and oligomers and/or create oligomers of higher molecular weight.

The degree of saturation of the third product may be increased by hydrogenating at least a portion of the third product extracted from fourth separator S4′ via line 246. At least a portion of the third product extracted from fourth separator S4′ via line 246 may be introduced into third reactor R3′. Suitable configurations and operating parameters for third reactor R3′ are provided hereinabove. Within third reactor R3′, hydrogen introduced via line 232 reacts with unsaturated components introduced via line 246, thus providing a more saturated or substantially entirely saturated product. In embodiments, the product of third reactor R3′, extracted therefrom via line 238, comprises saturated hydrocarbons, aromatic hydrocarbons and/or olefinic hydrocarbons, wherein the degree of unsaturation (or the olefinic content) of the product extracted via line 238 is less than or equal to the degree of unsaturation (or the olefinic content) of the feed introduced thereto via line 246. At least a portion of the product removed from third reactor R3′ via line 238 may be recycled to fourth reactor R4′ via line 242. Such recycle may serve to moderate one or more fourth reactor R4′ operating condition, such as, but not limited to, temperature, pressure and WHSV. In such embodiments, non-recycled product may be extracted via line 240.

In embodiments, the portion of third product from fourth separator S4′ that is not returned to fourth reactor R4′ as a processing aid and/or to enhance the overall process conversion is conveyed to third reactor R3′ wherein it is reacted with hydrogen introduced thereto via line 232 to form product which is removed from third reactor R3′ via line 238. The product removed from third reactor R3′ via line 238 comprises an increased portion of saturated hydrocarbons due to hydrogenation of reactive olefins and/or other unsaturated compounds.

The liquid hydrocarbons produced via this embodiment of the method may include, without limitation, a product in line 252 comprising olefinic naphtha and lighter olefinic and saturated hydrocarbons, and a product in line 240 comprising naphtha, gasoline, kerosene and heavier oil. In embodiments, the product in line 252 may be quantifiably differentiated by one or more of physical density, energy density, aromaticity, boiling point, olefin content and saturate content (i.e. degree of saturation) from the product in line 240. In embodiments, the process is operable to produce at least one product comprising between about 4 and about 90 volume percent aromatics, between about 10 and about 80 volume percent aromatics, between about 30 and about 70 volume percent aromatics, between about 20 and about 50 volume percent aromatics, between about 12 and about 20 volume percent aromatics, between about 8 and about 12 volume percent aromatics, or between about 4 and about 8 volume percent aromatics.

Process Comprising Three Oligomerizations. Another process for the production of liquid fuel from non-hydrocarbon components according to this disclosure will now be described with reference to FIG. 3. A feed comprising oxygen-containing compounds is converted into a product comprising primarily olefins by introduction thereof via line 310 into first reactor R1″. The configuration and operation of first reactor R1″ are discussed hereinabove. The feed may comprise one or more oxygen-containing compounds selected from C1 and higher alcohols, water, C2 and similar oxygenates including, but not limited to, acids, ethers, epoxides, aldehyes and other oxygen containing compounds. In embodiments, the primarily olefin product produced via first reactor R1″ comprises at least 50% of the available carbon as olefin product, at least 90% of the available feed carbon as olefin product, or at least 95% of the available feed carbon as olefin product. The primarily olefin product of first reactor R1″ may further comprise one or more components selected from alcohols, ethers, acetaldehyde, aldehydes, water, carbon monoxide, carbon dioxide, hydrogen, and C1 through C16 hydrocarbons.

The primarily olefin product of first reactor R1″ is introduced into first separator S1″ via line 312. The configuration and operation of first separator S1″ are described hereinabove. A liquid phase comprising primarily water, heavier oxygenates and C4+ hydrocarbons may be removed from first separator S1″ via line 314, and a gas phase comprising primarily olefins may be removed from first separator S1″ via line 320. In embodiments, the vapor phase removed from first separator S1″ via line 320 comprises at least about 20% of the available olefins, at least about 75% of the available olefins and at least about 90% of the available olefins.

The liquid product extracted from first separator S1″ via line 314 may be further separated into a water phase, a liquid hydrocarbon phase, and a light vapor stream. Such separation may be performed via a second separator S2″, as described hereinabove. A water phase may be removed from second separator S2″ via line 318. A liquid hydrocarbon phase may be removed from second separator S2″ via line 330. In embodiments, the liquid hydrocarbon phase removed from second separator S2″ via line 330 comprises at least 50 volume percent hydrocarbons, at least 80 volume percent hydrocarbons, or at least 95 volume percent hydrocarbons. In embodiments, hydrocarbons in the liquid hydrocarbon phase separated from separator S2″ via line 330 may be further processed by introduction into reactor R2″ and/or reactor R4″ via, for example third separator S3″.

A light vapor stream comprising oxygenates may be extracted from second separator S2″ via line 316. In embodiments, the vapor phase removed from second separator S2″ via line 316 comprises at least 5 volume percent oxygenates, at least 10 volume percent oxygenates, or at least 50 volume percent oxygenates. In such embodiments, oxygenates in the light vapor stream may be returned to first reactor R1″ via line 316 for reprocessing.

In embodiments, non-olefins are removed from the vapor phase separated via first separator S1″. For example, the vapor phase separated from first separator S1″ via line 320 may be introduced into gas cleanup apparatus GC1″, whereby non-olefins may be removed. In embodiments, substantially all of the non-olefins are removed from the vapor phase extracted from first separator S1″ via line 320, providing a purified olefin stream. In embodiments, a purified olefin product is removed from gas cleanup apparatus GC1″ via line 324. In embodiments, the purified olefin product comprises less than about 5 volume percent non-olefin components, less than about 0.5 volume percent non-olefin components, or less than about 0.01 volume percent non-olefin components. Primarily non-olefin components may be removed from gas cleanup apparatus GC1″ via line 322.

A portion of the olefins within the purified olefin product extracted from gas cleanup apparatus GC1″ may be converted primarily to oligomers within second reactor R2″. A portion of the purified olefin product extracted from GC1″ may be introduced into second reactor R2″ via lines 324 and 325. The configuration and operation of second reactor R2″ are described hereinabove. Second reactor R2″ may provide a product, extractable via line 326, comprising primarily oligomers. The product of second reactor R2″ may further comprise minor amounts of aromatics and/or other hydrocarbons. In embodiments, the product of second reactor R2″ comprises one or more components selected from dimers and oligomers of the olefins introduced thereto, mono- and multiply substituted aromatic compounds, isomerized olefins, saturated hydrocarbons, naphthenes, saturated and unsaturated cyclic hydrocarbons, and unreacted feed components. In embodiments, the product extracted from second reactor R2″ comprises primarily butenes and/or hexenes. In embodiments, the product extracted from second reactor R2″ via line 326 comprises ethylene, propylene, butylenes, pentenes, hexenes and/or higher mono-olefins. In embodiments, the product extracted from second reactor R2″ via line 326 comprises at least 20, 30, 40, 50, or 60 volume percent butylenes and/or heavier olefins.

A portion of the olefins within the purified olefin product extracted from gas cleanup apparatus GC1″ may be converted primarily to larger olefins, aromatics and/or other hydrocarbons within fifth reactor R5″. A portion of the purified olefin product extracted from gas cleanup apparatus GC1″ may be introduced into fifth reactor R5″ via lines 324 and 358. Suitable configurations and operating parameters for fifth reactor R5″ are provided hereinabove. Fifth reactor R5″ is fluidly connected with gas cleanup GC1″ via lines 324 and 358, whereby a portion of the purified olefin stream extracted from gas cleanup apparatus GC1″ via line 324 may be introduced into fifth reactor R5″.

Fifth reactor R5″ may be operated to provide a product comprising components selected from oligomers of the olefins introduced thereto via line 358, mono and multiply substituted aromatic compounds, isomerized olefins, saturated hydrocarbons, naphthenes, saturated and unsaturated cyclic hydrocarbons, and/or unreacted feed components. In embodiments, the product of fifth reactor R5″, extracted therefrom via line 360, comprises at least about 50% of the available carbon as olefin product, at least about 90% of the available feed carbon as olefin product, or at least about 95% of the available feed carbon as olefin product. In embodiments, the product of fifth reactor R5″, extracted therefrom via line 360, comprises from about 4 volume percent to about 85% aromatics.

Unreacted, gaseous olefins may be removed from the product of fifth reactor R5″ via fifth separator S5″. Suitable configurations and operating parameters for fifth separator S5″ are provided hereinabove. Liquid hydrocarbons may be removed from fifth separator S5″ via line 366, and a gaseous/vapor phase comprising unreacted olefins may be removed from fifth separator S5″ via line 362. In embodiments, the liquid extracted via line 366 comprises liquid hydrocarbons, including, but not necessarily limited to, one or more components selected from oligomers of the primarily olefin feed to reactor R5″, mono- and multiply-substituted aromatic compounds, isomerized olefins, saturated hydrocarbons, naphthenes, and saturated and unsaturated cyclic hydrocarbons. Unreacted olefins removed from fifth separator S5″ via line 362 may be compressed via third compressor C3″ and reintroduced via line 364 into fifth reactor R5″ for further processing. In embodiments, the liquid product removed from fifth separator S5″ via line 366 comprises at least about 20% of the available olefins, at least about 75% of the available olefins, or about 90% of the available olefins. In embodiments, the liquid product removed from fifth separator S5″ via line 366 comprises at least 20 volume percent of the unsaturated hydrocarbons introduced thereto, at least 75 volume percent of the unsaturated hydrocarbons introduced thereto, or at least about 90 volume percent of the unsaturated hydrocarbons introduced thereto.

The degree of saturation of the liquid hydrocarbons removed from fifth separator S5″ may be increased by hydrogenation. For example, in embodiments, the liquid hydrocarbon product extracted from fifth separator S5″ via line 366 is introduced via line 366 into sixth reactor R6″. Suitable configurations and operating conditions for hydrogenation reactor R6″ are provided hereinabove. Within sixth reactor R6″, hydrogen introduced via line 368 reacts with unsaturated components of the feed introduced thereto via line 366, providing a liquid hydrocarbon product that is more saturated than the feed. The liquid hydrocarbon product removed from sixth reactor R6″ via line 370 comprises more saturated or substantially entirely saturated product. The product of sixth reactor R6″, extracted therefrom via line 370, may comprise one or more component selected from saturated hydrocarbons, aromatic hydrocarbons and olefinic hydrocarbons, wherein the degree of unsaturation (or the olefinic content) of the product extracted via line 370 is less than or equal to the degree of unsaturation (or the olefinic content) of the feed introduced thereto via line 366. The degree of unsaturation (e.g. the olefin content) of at least a portion of the product of sixth reactor R6″ may be reduced by introduction of at least a portion thereof into third reactor R3″ via lines 374 and/or 350. Non-recycled product may be removed (e.g. for storage and/or etc.) via line 372.

The liquid hydrocarbons removed from separator S5″ may also be processed jointly with the products of reactor R4″ by introducing them to separator S4″ via line 367.

Unreacted olefinic compounds may be removed from the product of second reactor R2″ via third separator S3″. Suitable configurations and operating parameters for third separator S3″ are provided hereinabove. Within third separator S3″, a liquid hydrocarbon stream may be separated from a vapor phase comprising primarily unreacted olefinic compounds. Liquid hydrocarbons may be removed from third separator S3′ via line 334, and a gaseous/vapor phase comprising unreacted olefins may be removed from third separator S3″ via line 336. In embodiments, the liquid extracted via line 334 comprises liquid hydrocarbons, including, but not necessarily limited to, dimers and oligomers of the primarily olefin feed to reactor R2″, mono- and multiply-substituted aromatic compounds, isomerized olefins, saturated hydrocarbons, naphthenes, and/or saturated and unsaturated cyclic hydrocarbons. Unreacted olefins removed from third separator S3″ via line 336 may be compressed via first compressor C1″ and reintroduced via line 328 into second reactor R2″ for further processing. In embodiments, the liquid product removed from third separator S3″ via line 334 comprises at least 20 volume percent oligomers, at least 50 volume percent oligomers, or at least 75 volume percent oligomers.

At least a portion of the olefin dimers and oligomers of the olefins that are produced in reactor R2″ may be converted to longer chain olefins. For example, in embodiments, the liquid product removed from third separator S3″ via line 334 may be introduced into fourth reactor R4″. Suitable configurations and operating conditions for fourth reactor R4″ are provided hereinabove. Within fourth reactor R4″, the average chain length of the olefins fed thereto is increased. Additionally, a portion of the reactive olefins introduced into fourth reactor R4″ may be converted to aromatic compounds, a portion may be isomerized to isoalkenes, a portion may be converted to cyclic compounds and/or a portion may undergo cracking and/or disproportioning to smaller saturated and/or unsaturated compounds.

In embodiments, the product of fourth reactor R4″ comprises higher oligomers comprising from C6 to C30+ hydrocarbons. In embodiments, the product of fourth reactor R4″ comprises olefinic naphtha, gasoline, middle distillates, heavier oil, and/or waxes.

The product of fourth reactor R4″ may be separated into a variety of products. For example, in embodiments, the product of fourth reactor R4″ is separated into three or more products via fourth separator S4″. A first product comprising olefinic vapor, a second product comprising one or more olefins selected from the group consisting of C2 through C20 olefins, and a third product comprising naphtha, middle distillates, and heavier oil may be separated from the feed introduced to fourth separator S4″ via line 344.

A first product may be extracted from fourth separator S4″ via line 354, the first product comprising olefinic products. The first product may comprise at least about 20 weight percent olefins at least about 50 weight percent olefins, or at least about 70 weight percent olefins.

A second product may be extracted from fourth separator S4″ via line 351, the second product comprising at least about 40 weight percent of one or more olefins selected from the group consisting of C2 through C20 olefins, at least about 60 weight percent of one or more olefins selected from the group consisting of C2 through C20 olefins, or at least about 80 weight percent of one or more olefins selected from the group consisting of C2 through C20 olefins.

A third product may be extracted from fourth separator S4″ via line 346, the third product comprising at least about 20 weight percent olefinic naphtha, middle distillates, and/or heavier oil, at least about 40 weight percent olefinic naphtha, middle distillates, and/or heavier oil, or at least about 70 weight percent olefinic naphtha, middle distillates, and/or heavier oil.

All or a portion of the first product comprising olefins removed from fourth separator S4″ via line 354 may be recycled via second compressor C2″ and line 359 to second reactor R2″, via second compressor C2″ and line 356 to fourth reactor R4″, or both.

Optionally, a portion of the second product, comprising one or more olefins selected from the group consisting of C2 through C20 olefins, removed from fourth separator S4″ via line 351 may be may be returned to fourth reactor R4″ via, for example, line 348, pump P1″ and line 358. Such recycle of a portion of the third product to the fourth reactor R4″ may enable improved processing and/or higher conversion of olefins to olefin dimers and oligomers and/or create oligomers of higher molecular weight.

The degree of saturation of the third product may be increased by hydrogenating at least a portion of the third product extracted from fourth separator S4″ via line 346. At least a portion of the third product extracted from fourth separator S4″ via line 346 may be introduced into third reactor R3″. Suitable configurations and operating parameters for third reactor R3″ are provided hereinabove. Within third reactor R3″, hydrogen introduced via line 332 reacts with unsaturated components introduced via line 346, thus providing a more saturated or substantially entirely saturated product. In embodiments, the product of third reactor R3″, extracted therefrom via line 338, comprises saturated hydrocarbons, aromatic hydrocarbons and/or olefinic hydrocarbons, wherein the degree of unsaturation (or the olefinic content) of the product extracted via line 338 is less than or equal to the degree of unsaturation (or the olefinic content) of the feed introduced thereto via line 346. At least a portion of the product removed from third reactor R3″ via line 338 may be recycled to fourth reactor R4″ via line 342. Such recycle may serve to moderate one or more fourth reactor R4″ operating condition, such as, but not limited to, temperature, pressure and WHSV. In such embodiments, non-recycled product may be extracted via line 340.

In embodiments, the portion of third product from fourth separator S4″ that is not returned to fourth reactor R4″ as a processing aid and/or to enhance the overall process conversion is conveyed to third reactor R3″ wherein it is reacted with hydrogen introduced thereto via line 332 to form product which is removed from third reactor R3″ via line 338. The product removed from third reactor R3″ via line 338 comprises an increased portion of saturated hydrocarbons due to hydrogenation of reactive olefins and/or other unsaturated compounds.

The liquid hydrocarbons produced via this embodiment of the method may include, without limitation, a product in line 352 comprising olefinic naphtha and lighter olefinic and saturated hydrocarbons, a product in line 340 comprising gasoline, middle distillate and heavier oil, and a product in line 372 comprising gasoline, middle distillate and heavier oil. In embodiments, the products in lines 351, 372, and/or 340 may be quantifiably differentiated by one or more of physical density, energy density, aromaticity, boiling point, olefin content and saturate content (i.e. degree of saturation).

Features. The disclosed system and method allow, via the use of multiple reactors and adjustable amounts of recycle of various components thereto, for the production of a fully designable product, i.e. a product having a desired aromaticity, degree of saturation, and/or average molecular weight of hydrocarbons. For example, the disclosed system and method allow for the production of jet fuel or jet fuel components having a desired aromatics content, or the production of primarily diesel, gasoline, kerosene, or naphtha products. In embodiments, the disclosed system and method are operable to produce at least one product comprising between about 10 and about 90 volume percent aromatics, between about 20 and about 80 volume percent aromatics, between about 30 and about 70 volume percent aromatics, between about 20 and about 50 volume percent aromatics, between about 12 and about 20 volume percent aromatics, between about 8 and about 12 volume percent aromatics, between about 4 and about 8 volume percent aromatics, less than 25 volume percent aromatics, less than 18 volume percent aromatics, less than 15 volume percent aromatics, or less than 12 volume percent aromatics. In embodiments, the disclosed system and method incorporate at least two oligomerization reactors and are operable to provide a moderate aromaticity (i.e. in the range of from about 4 to about 90 weight percent aromatic compounds) product by operating a first oligomerization reactor to convert ethylene to a first product comprising primarily butene and substantial octene; and a second oligomerization reactor to oligomerize the butene and octene to provide higher oligomers that comprise C6 through C30+.

In embodiments, the disclosed system and method enable the production of at least one high aromaticity product (i.e. in the range of from about 25 to about 90 weight percent aromatic compounds) and at least one low aromaticity (i.e. in the range of from about 4 to about 20 weight percent aromatic compounds) or moderate aromaticity (i.e. in the range of from about 8 to about 45 weight percent aromatic compounds) product that may be combined to make a product of intermediate aromatic content (i.e. in the range of from about 4 to about 90 weight percent aromatic compounds) and properties commensurate with the composition and boiling point range of the mixture.

In embodiments, the disclosed system and method enable the production of at least one low aromaticity (i.e. in the range of from about 4 to about 20 weight percent aromatic compounds) product and at least one moderate aromaticity (i.e. in the range of from about 8 to about 45 weight percent aromatic compounds) product that may be combined to provide a product of intermediate aromaticity (i.e. in the range of from about 4 to about 45 weight percent aromatic compounds) and concomitant properties.

In embodiments, the disclosed system and method incorporate one or more recycles, whereby substantially all of the olefins in an olefin feed may be reacted to produce substantially non-reactive alkanes and/or aromatic compounds.

In embodiments, the disclosed system and method incorporate two or more reactors that oligomerize butene-containing streams, whereby a feed comprising butene may be split advantageously between the two reactors to make more or less aromatic product by volume, as desired. In embodiments, the disclosed system and method incorporate two or more butene oligomerization reactors operated at different conditions of pressure, temperature and/or WHSV that oligomerize butene-containing streams such that advantageously splitting of the butene feed to the two reactors may provide for a more or less aromatic product by volume, as desired. The two or more butene-oligomerization reactors may utilize the same catalyst or different catalysts.

In embodiments, the disclosed system and method incorporate two or more reactors, operable at different conditions of pressure, temperature and/or WHSV that oligomerize ethylene-containing streams, whereby a feed comprising ethylene may be split advantageously between the two reactors to provide more or less oligomer product by volume from the combined process. The ethylene oligomerization reactors may utilize the same catalyst or different catalysts. The oligomer product formed from the ethylene may comprise butenes, hexenes, octenes, decenes, heavier mono-olefins, oligomers, naphthenes and/or aromatic components.

While the preferred embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

To further illustrate various illustrative embodiments of the present invention, the following examples are provided.

### EXAMPLES

### Example 1

### Production of Liquid Hydrocarbons via Single Oligomerization Reactor

A solution of ethanol (95 volume percent) and water is fed to a first reactor filled with aluminum oxide to produce a product comprising ethylene as well as water (H₂O), carbon monoxide (CO), carbon dioxide (CO₂), and organic oxygenates. Table A shows the composition of the product comprising ethylene.

TABLE A
Analysis of Ethylene made from Ethanol

| | |
|---|---|
| Ethylene, weight percent | 99.5+ |
| Oxygen, ppm | 1.8 |
| Nitrogen, ppm | 14.3 |
| Carbon Monoxide, ppm | <1 |
| Carbon Dioxide, ppm | <1 |
| Acetylene, ppm | <1 |
| Methane, ppm | 7.7 |
| Ethane, ppm | 420.9 |
| Sulfur, ppm | <1 |
| Moisture, ppm | 2 |

The water, carbon monoxide and organic oxygenates are removed from the ethylene in this case by use of separators selected from absorbent beds comprising molecular sieves, granulated active carbon, CDX (silica gel) and R3-11G (carbon based copper catalyst). Although these media were used in this example to purify the stream comprising ethylene, there are many other materials that are known by those skilled in the art used to remove contaminants such as these as well as other processing methods, such as cryogenic processing, gas sweetening, and distillation. The ethylene rich stream whose composition is depicted in Table A was then fed, under adiabatic conditions, to a second reactor containing ZSM-5 catalyst. The pressure was maintained at 400 psig and the temperature was measured to range from 300° C. to 320° C. The product of this second reactor was sent to a third (hydrogenation) reactor which contained Ni/Mo on alumina. The middle distillate portion of the product of this reaction has a very high aromatic content, as shown in Table B.

TABLE B
Analysis of Product made from Ethylene in One Reactor

| | |
|---|---|
| Physical Density, g/mL @ 60° | 0.8765 |
| Energy Density, BTU/lb | 17896 |
| Initial BP, ° F. | 310 |
| Final BP, ° F. | 600 |
| Flash Point, ° F. | 110 |
| Freeze Point, ° C. | <−65 |
| Aromatic Content, vol. % | 82.7 |
| Olefin Content, vol. % | 0.5 |
| Saturate Content, vol. % | 16.8 |

### Example 2

### Production of Liquid Hydrocarbons via Dual Oligomerization Reactors

A solution of ethanol (95 volume percent) with water is fed to a first reactor filled with aluminum oxide to produce some ethylene as well as water (H₂O), carbon monoxide (CO), carbon dioxide (CO₂), and organic oxygenates. Table A shows the composition of the product comprising ethylene. The water, carbon monoxide and organic oxygenates are removed from the ethylene in this case by use of separators including absorbent beds comprising molecular sieves, granulated active carbon, CDX (silica gel) and R3-11G (carbon based copper catalyst). Although these media were used in this example to purify the stream comprising ethylene, there are many other materials that are known by those skilled in the art used to remove contaminants such as these as well as other processing methods, such as cryogenic processing, gas sweetening, and distillation. The ethylene was sent to a second (or ‘primary oligomerization’) reactor that contained a catalyst comprising 3% Ni imbibed onto ZSM-5 catalyst. The second reactor was held at temperatures in the range of from 220° C. to 240° C. under temperature control and a pressure of between 300 and 400 psia. The second reactor was effective at converting 25 mole percent of the ethylene to butene or heavier olefins as shown in Table C.

TABLE C
Results of Reactive Conversion of Ethylene to Butylene

| | |
|---|---|
| Ethylene Feed Rate, g/h | 70.5 |
| Ni/ZSM-5 Catalyst, g | 160 |
| WHSV, hr⁻¹ | 0.440625 |
| Days on Stream | 5 |
| Reactor Inlet Pressure, psig | 322 |
| Reactor Outlet Pressure, psig | 316 |
| Reactor Peak Temperature, ° C. | 244 |
| Ethylene Conversion, mole % | 26.20 |
| Propylene, carbon % | 0.50 |
| Butylene Selectivity, carbon % | 76.50 |
| C5 Olefins, carbon % | 1.80 |
| C6 Olefins Selectivity, carbon % | 6.40 |
| Total C3-C6 Olefins, carbon % | 85.20 |
| Paraffins, carbon % | 2.00 |
| Liquids | None |

The product of the primary oligomerization reactor was sent to a third (or ‘secondary oligomerization’) reactor operated under temperature control, containing a nickel on ZSM-5 catalyst. The secondary oligomerization reactor was operated at temperatures between 240° C. and 300° C. and at pressures between 200 psia and 400 psia. Table D presents data that indicate that the aromatic content of the final product is strongly dependent upon the temperatures in both oligomerization reactors.

TABLE D
Aromatic Content of Fuel Produced via Conversion of Ethylene via Dual Oligomerization Reactors

| | | | | | | | Hydrogenated | Hydrogenated |
|---|---|---|---|---|---|---|---|---|
| Olig. 1 | Olig. 1 | Olig. 2 | Olig. 2 | Raw Aromatic | Raw Saturate | | Aromatic | Saturate |
| Temp., ° C. | Pressure, ° C. | Temp., ° C. | Pressure, ° C. | Content, vol % | Content, vol % | Raw SG | Content, vol % | Content, vol % |
| 220 | — | 251 | — | 12.9 | 48.50 | 0.783 | — | — |
| 220 | 329 | 258 | 220 | 19.2 | 4.30 | — | — | — |
| 220 | 329 | 258 | 220 | 5.20 | 93.70 | — | — | — |
| 220 | — | 260 | — | 13.1 | 46.30 | 0.780 | — | — |
| 220 | — | 260 | — | 6.10 | 37.10 | 0.783 | 9.60 | 89.80 |
| 226 | 319 | 254 | 217 | 5.70 | 31.40 | 0.787 | 9.40 | 90.60 |
| 229 | 317 | 256 | 215 | 9.10 | 26.20 | 0.788 | 10.00 | 89.50 |
| 230 | 302 | 255 | 216 | 54.50 | 23.50 | 0.778 | — | — |
| 237 | 319 | 262 | 228 | 8.9 | — | — | — | — |
| 239 | 317 | 280 | 223 | 49.0 | 30.30 | — | — | — |
| 240 | 313 | 260 | 212 | 2.5 | 3.90 | — | 5.7 | 94.30 |
| 240 | 319 | 278 | 212 | 18.10 | 29.10 | 0.798 | 16.30 | 83.50 |
| 243 | 324 | 273 | 214 | 12.30 | 26.00 | 0.798 | 10.50 | 88.50 |
| 243 | 313 | 278 | 212 | 11.6 | 5.70 | — | — | — |
| 243 | — | 278 | — | 7.3 | 5.90 | — | 3.50 | 95.50 |
| 245 | 341 | 275 | 250 | 41.2 | 14.40 | — | — | — |
| 247 | 341 | 286 | 230 | 9.6 | 8.10 | — | — | — |
| 247 | 333 | 300 | 245 | 8.0 | 4.10 | — | — | — |
| 247 | 333 | 300 | 230 | — | — | — | 6.0 | 92.40 |
| 251 | 336 | 295 | 240 | 8.5 | 4.40 | — | — | — |

While the preferred embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of a reference herein is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

What is claimed is:

1. A method comprising:
providing an olefin feed comprising at least one olefin selected from the group consisting of C2-C20 olefins;
oligomerizing at least a part of the olefin feed in the presence of a first oligomerization catalyst to form a first oligomerization product comprising oligomers of the at least one olefin; and
subjecting at least a portion of the first oligomerization product to oligomerization in the presence of a second oligomerization catalyst to produce a second oligomerization product.

2. The method of claim 1 further comprising hydrogenating at least a portion of the second oligomerization product to produce a hydrogenation product, wherein the average degree of saturation of the hydrogenation product is greater than the average degree of saturation of the second oligomerization product.

3. The method of claim 2 wherein the hydrogenation product comprises primarily hydrocarbons boiling in a boiling point range selected from the group consisting of the gasoline boiling point range, the naphtha boiling point range, the kerosene boiling point range and the diesel boiling point range.

4. The method of claim 3 wherein the hydrogenation product comprises from about 20 to about 50 volume percent aromatic compounds.

5. The method of claim 3 wherein the hydrogenation product comprises from about 8 to about 12 volume percent aromatic compounds.

6. The method of claim 3 wherein the hydrogenation product comprises from about 4 to about 8 volume percent aromatic compounds.

7. The method of claim 3 wherein the hydrogenation product comprises from about 12 to about 20 volume percent aromatic compounds.

8. The method of claim 1 wherein providing the olefin feed comprises performing one or more process selected from the group consisting of alcohol dehydration processes and acetylenic compound hydrogenation processes.

9. The method of claim 1 wherein providing the olefin feed comprises catalytically converting an oxygen-containing compound in an oxygenate feed to an olefin.

10. The method of claim 9 wherein the olefin feed comprises ethylene and wherein providing the olefin feed further comprises separating a stream enriched in ethylene from the product of the catalytic conversion of the oxygenate feed.

11. The method of claim 10 further comprising separating a stream comprising unreacted oxygen-containing compounds from an ethylene-reduced remainder of the product of the catalytic conversion of the oxygenate feed.

12. The method of claim 11 further comprising producing additional olefin from the stream comprising unreacted oxygen-containing compounds.

13. The method of claim 11 further comprising separating a liquid hydrocarbon phase, a water phase, or both from the ethylene-reduced remainder of the product of the catalytic conversion of the oxygenate feed.

14. The method of claim 13 further comprising subjecting at least a portion of the liquid hydrocarbon phase to the second oligomerization.

15. The method of claim 1 further comprising separating an unreacted olefin stream comprising unreacted olefin from the first oligomerization product prior to the second oligomerization.

16. The method of claim 15 further comprising recycling at least a portion of the unreacted olefin stream to the first oligomerization.

17. The method of claim 1 further comprising separating the second oligomerization product into one or more streams selected from the group consisting of a stream enriched in olefins boiling in the gasoline boiling point range, a stream enriched in naphtha, and a stream comprised of the remaining C2 through C20 olefins relative to the second oligomerization product.

18. The method of claim 17 further comprising recycling at least a portion of the stream enriched in olefins boiling in the gasoline boiling point range to the first oligomerization, the second oligomerization, or both.

19. The method of claim 17 further comprising subjecting at least a portion of the stream enriched in C2 through C20 olefins to hydrogenation to produce a hydrogenation product, wherein the average degree of saturation of the hydrogenation product is greater than the average degree of saturation of the stream enriched in C2 through C20 olefins.

20. The method of claim 19 further comprising recycling at least a portion of the hydrogenation product to the second oligomerization.

21. The method of claim 17 further comprising subjecting at least a portion of the stream enriched in C2 through C20 olefins to the second oligomerization.

22. The method of claim 1 further comprising subjecting a portion of the first oligomerization product to oligomerization in the presence of a third oligomerization catalyst to produce a third oligomerization product.

23. The method of claim 22 further comprising separating an unreacted olefin stream comprising unreacted olefin from the third oligomerization product.

24. The method of claim 23 further comprising recycling at least a portion of the unreacted olefin stream to the third oligomerization.

25. The method of claim 22 further comprising subjecting at least a portion of the third oligomerization product at least a portion of the second oligomerization product, or both to hydrogenation to produce one or more hydrogenation products, wherein the average degree of saturation of the one or more hydrogenation products is greater than the average degree of saturation of the oligomerization product prior to hydrogenation.

26. The method of claim 25 further comprising subjecting at least a portion of the one or more hydrogenation products to the second oligomerization.

27. The method of claim 1 wherein the first oligomerization catalyst comprises nickel activated solid acid catalyst.

28. The method of claim 27 wherein the second oligomerization catalyst comprises a solid acid catalyst.

29. The method of claim 1 wherein the first oligomerization product comprises primarily butene and the second oligomerization product comprises oligomers of butene.

30. A system comprising:
a first oligomerization reactor configured to oligomerize at least a portion of an olefin feed comprising at least one olefin in the presence of a first oligomerization catalyst, providing a first oligomerization product;
a separator configured to separate unreacted olefin from the first oligomerization product, providing an unreacted olefin reduced first oligomerization product; and
a second oligomerization reactor configured to oligomerize at least a portion of the unreacted olefin reduced first oligomerization product in the presence of a second oligomerization catalyst, providing a second oligomerization product.

31. The system of claim 30 wherein the olefin feed comprises primarily ethylene.

32. The system of claim 30 wherein the olefin feed comprises primarily butene.

33. The system of claim 30 wherein the first oligomerization catalyst is operable to convert the at least one olefin primarily to dimers thereof.

34. The system of claim 33 wherein the first oligomerization catalyst comprises nickel activated solid acid catalyst.

35. The system of claim 30 wherein the second oligomerization catalyst is operable to convert at least a portion of oligomers in the first oligomerization product to longer chain olefins.

36. The system of claim 35 wherein the second oligomerization catalyst comprises a solid acid catalyst.

37. The system of claim 30 further comprising a hydrogenation reactor configured to catalytically reduce the degree of unsaturation of the second oligomerization product, providing a hydrogenated product.

38. The system of claim 37 further comprising recycling at least a portion of the hydrogenated product, at least a portion of the second oligomerization product, or both, to the first oligomerization reactor, the second oligomerization reactor, or both, whereby a hydrocarbon product having a desired composition may be obtained.

39. The system of claim 38 wherein the desired composition comprises an aromatics content in the range of from about 4 to about 50 volume percent.

40. The system of claim 39 wherein the desired composition comprises an aromatics content in the range of from about 4 to about 8 volume percent.

41. The system of claim 39 wherein the desired composition comprises an aromatics content in the range of from about 8 to about 12 volume percent.

42. The system of claim 39 wherein the desired composition comprises an aromatics content in the range of from about 12 to about 20 volume percent.

43. The system of claim 38 wherein the desired composition comprises primarily diesel, kerosene, gasoline, or naphtha boiling point range hydrocarbons.

44. The system of claim 30 further comprising a second separator configured to separate one or more streams selected from the group consisting of a stream comprising primarily naphtha, a stream comprising primarily gasoline, and a stream comprising primarily C2-C30 olefins from the second oligomerization product.

45. The system of claim 44 further comprising one or more recycle lines configured for recycle of at least a portion of a stream comprising primarily gasoline to the first oligomerization reactor, to the second oligomerization reactor, or to both.

46. The system of claim 44 further comprising a recycle line configured for recycle of at least a portion of a stream comprising primarily C2-C30 olefins to the second oligomerization reactor.

47. The system of claim 30 further comprising a third oligomerization reactor configured to oligomerize at least a portion of an olefin feed comprising at least one olefin in the presence of a third oligomerization catalyst, providing a third oligomerization product.

48. The system of claim 47 wherein the third oligomerization catalyst comprises a solid acid catalyst.

49. The system of claim 47 further comprising a second separator configured to remove unreacted olefins from the third oligomerization product, providing an unreacted olefin reduced third oligomerization product.

50. The system of claim 49 further comprising a recycle line configured for recycle of at least a portion of the unreacted olefins removed via the second separator to the second oligomerization reactor.

51. The system of claim 47 further comprising one or more hydrogenation reactors configured to catalytically reduce the degree of unsaturation of the second oligomerization product, the third oligomerization product or both, providing one or more hydrogenated products.

52. The system of claim 51 further comprising one or more recycle lines whereby at least a portion of the one or more hydrogenated products may be recycled to the second oligomerization reactor, the third oligomerization reactor, or both.

53. The system of claim 30 further comprising a dehydration reactor configured to produce the olefin feed.

54. The system of claim 53 wherein the dehydration reactor is operable at a pressure greater than an operating pressure of the first oligomerization reactor, and wherein the system comprises no compressor between the dehydration reactor and the first oligomerization reactor.
